(19) United States
(12) Patent Application Publication
(10) Pub. No.: US 2026/0273022 A1
(43) Pub. Date: Sep. 17, 2026

(54) GLP-1R and GCGR Agonist in the Treatment of Alcohol Use Disorder (AUD) in Subjects with Obesity or Overweight Human Beings

(71) Applicant: Spitfire Pharma LLC, Gaithersburg, MD (US)

(72) Inventors: Bertrand Victor Gilbert Georges, Stevenage (GB); M. Scot Roberts, Bethesda, MD (US); Sarah K. Browne, Chevy Chase, MD (US); Matthew Scott Harris, Washington, DC, DC (US); John Joseph Suschak, III, Damascus, MD (US)

(73) Assignee: Spitfire Pharma LLC, Gaithersburg, MD (US)

(21) Appl. No.: 19/566,403

(22) Filed: Mar. 13, 2026

Related U.S. Application Data
(60) Provisional application No. 63/771,184, filed on Mar. 13, 2025.

(51) Int. Cl. A61K 38/26; A61K 31/145; A61K 31/185; A61K 31/485; A61K 45/06; A61P 3/04; A61P 25/32

(52) U.S. Cl. CPC A61K 38/26; A61K 31/145; A61K 31/185; A61K 31/485; A61K 45/06; A61P 3/04; A61P 25/32

(57) ABSTRACT

This disclosure relates to the once weekly dosing regimen of a dual GLP-1R and GCGR agonist, formulations, and methods of using the same for treatment of Alcohol Use Disorder (AUD) and/or alcohol dependence using a pharmaceutical dosage formulation comprising a peptide product according to SEQ ID NO: 1, or peptide product that is at least 80% identical to a peptide of SEQ ID NO: 1.

### RELATED APPLICATIONS

This application claims priority to U.S. Ser. No. 63/771,184 filed on Mar. 13, 2025, which is incorporated by reference in its entirety into this application.

### SEQUENCE LISTING

The instant application contains a Sequence Listing which has been submitted electronically in ASCII format via the USPTO Patent Center and hereby incorporated by reference in its entirety. The ASCII copy, created on 1 Mar. 2026, is named MED015_20260301.XML and is 2,000 bytes in size.

### FIELD OF THE DISCLOSURE

This disclosure relates to the field of GLP-1R and GCGR agonists, formulations, and methods of using the same to prevent and/or treat Alcohol Use Disorder in a human being.

### BACKGROUND OF THE DISCLOSURE

Alcohol use disorder (AUD) is a major public health problem with an estimated 60 million binge drinkers in the United States (US) (i.e., ≥5 standard drinks for males and ≥4 standard drinks for females on an occasion). In addition, an estimated 12.2% of the US population engaged in heavy drinking within the prior month according to the National Survey on Drug Use and Health from 2021. In addition to its substantial negative impact on economic, societal and healthcare costs, AUD can lead to significant health implications, affecting many organs particularly the liver but also the brain, gut, pancreas and the lungs. A leading complication of AUD involves progressive damage to the liver, commonly grouped under the umbrella of alcohol-related liver disease (ARLD), which can range from benign steatosis to advanced fibrosis. Alcohol-related conditions are the fourth leading preventable cause of death.

Currently approved AUD medications vary by region: disulfiram, acamprosate, and naltrexone are approved by both the U.S. Food and Drug Administration (FDA) and the European Medicines Agency (EMA), nalmefene by the EMA, and baclofen by the French Medicines Agency. Other drugs (e.g., gabapentin, topiramate, varenicline) are recommended for off-label use. Despite evidence supporting their efficacy, these medications remain severely underutilized; for example, fewer than 2% of people with AUD in the United States receive approved pharmacotherapies, with similarly low rates in Canada (<0.4%) and Australia (<3%). Contributing factors include limited physician training, concerns about drug contraindications (e.g., naltrexone or disulfiram in severe liver disease, acamprosate in kidney impairment, or disulfiram in cognitive impairment), the need for close adherence monitoring, and variable patient responses linked to genetics and sex-based differences in safety and efficacy. Moreover, relapse rates can be as high as 45-90% in the first three years, influenced by patient characteristics (e.g., age, health status, and motivation) and psychosocial stressors. These challenges underscore an urgent need for novel therapeutic approaches.

Studies in rodents and non-human primates have shown that GLP-1 agents reduce alcohol intake and decrease relapse-like alcohol drinking. GLP-1 may target areas associated with alcohol craving and addiction in the brain. In a recent clinical trial, liraglutide led to decreased alcohol consumption in individuals with type 2 diabetes mellitus (T2D). Real-world data derived from social media posts in patients with overweight or obesity on semaglutide or tirzepatide show a significant reduction in self-reported intake of alcohol consumption, drinks per drinking episode, binge drinking odds, and Alcohol Use Disorders Identification Test (AUDIT) scores. In a retrospective cohort study analyzing electronic health records if over 800,00 patients with a history of AUD, GLP-1 receptor agonists (RAs) were associated with lower rates of alcohol intoxication in patients with established AUD. The protective effects were consistent across patient subgroups including those with T2D and obesity. Although a clinical trial of exenatide in individuals seeking treatment for AUD did not significantly reduce days of heavy drinking in the experimental group, functional magnetic resonance imaging (fMRI) revealed significant attenuation of activity in the ventral striatum and septal area when images of alcohol were shown. Further analysis also revealed a substantial reduction in heavy drinking in patients with obesity (body mass index [BMI] ≥30 kg/m²) in the experimental group. Importantly, no in vitro drug-drug interactions (DDIs) between GLP-1 agents and alcohol metabolizing enzymes or adverse clinical interactions between GLP-1 and alcohol use have been reported.

While GLP-1 receptor (GLP-1R) agonists alone have shown promise in mitigating alcohol cravings and reducing alcohol intake, the concurrent activation of the glucagon receptor (GCGR) may provide synergistic benefits for AUD. GCGR agonism can enhance metabolic homeostasis by promoting hepatic lipid oxidation and energy expenditure, thus potentially counteracting the caloric load of alcohol consumption and helping to minimize alcohol-related metabolic stress on the liver. This combination strategy may not only address key metabolic risk factors (e.g., obesity, fatty liver and/or dyslipidemia), which often co-exist in individuals with AUD, but also holds the potential to yield more robust, sustained reductions in alcohol-seeking behavior compared to GLP-1R agonists alone.

Methods for treating Alcohol Use Disorders (AUDs) are lacking and desired by those in the field. This disclosure provides such methods using pemvidutide, as described below.

### SUMMARY OF THE DISCLOSURE

Described herein are methods for the treatment of Alcohol Use Disorder (AUD), including preventing and/or treating alcohol dependence, in a human being in need thereof, optionally including any related disorders, with peptide products of this disclosure. In some embodiments, this disclosure provides pharmaceutical dosage formulation of pemvidutide configured to prevent and/or treat one or more of alcohol dependencies and/or alcohol use disorders in a human being.

In embodiments provided herein are methods for treatment of alcohol use disorder (AUD) and/or alcohol dependence in a human being in need thereof comprising administering to the human being a once weekly therapeutic effective amount of a pharmaceutical dosage formulation comprising a peptide product according to SEQ ID NO: 1, or a peptide product that is at least 80% identical to a peptide of SEQ ID NO: 1. In embodiments provided herein are methods for for reducing alcohol consumption and/or maintaining reduced alcohol consumption in treatment of alcohol use disorder (AUD) in a human being in need thereof comprising administering to the human being a once weekly therapeutic effective amount of a pharmaceutical dosage formulation comprising a peptide product according to SEQ ID NO: 1, or peptide product that is at least 80% identical to a peptide of SEQ ID NO: 1. In certain embodiments provided herein are methods for prevention of relapse to alcohol dependence in a human being in need thereof comprising administering to the human being a once weekly therapeutic effective amount of a pharmaceutical dosage formulation comprising a peptide product according to SEQ ID NO: 1, or peptide product that is at least 80% identical to a peptide of SEQ ID NO: 1.

In embodiments, the human being in need thereof has a co-morbidity selected from one or more of fatty liver disease, hypertension, dyslipidemia, type 2 diabetes, polycystic ovary syndrome (PCOS), cardiovascular disease, hyperglycemia, and hyperinsulinemia.

In embodiments, the treatment wherein the human being suffers from alcohol-related liver disease (ARLD); reduces excess body weight; reduces alcohol consumption by the human being; maintains reduced alcohol consumption by the human being; reduces phosphatidylethanol (PEth) levels in the human being; reduces liver enzymes alanine aminotransferase (ALT) and/or aspartate aminotransferase (AST) in the human being and/or reduces liver fat. In certain embodiments, the treatment reduces a symptom of withdrawal or prevents a relapse of the alcohol use disorder in the human, reduces tolerance to alcohol, reduces dependence on alcohol, improves adherence to a treatment for alcohol abuse, reduces a preference for alcohol, decreases liking for alcohol, or increases abstinence from alcohol.

In embodiments, human being is obese or overweight. In embodiments, the human being in need thereof has a body mass index (BMI kg/m²) of at least 25; has a body mass index (BMI kg/m²) of at least 30; has a body mass index (BMI kg/m²) of at least 35; or has a body mass index (BMI kg/m²) of at least 40.

In embodiments the peptide product is at least 85%, at least 90%, at least 95%, at least 96%, at least 97%, at least 98% or at least 99% identical to a peptide of SEQ ID NO: 1. In exemplary embodiments provided herein is peptide product that is 100% identical to a peptide of SEQ ID NO: 1 for use in methods of this disclosure.

In embodiments, the pharmaceutical dosage formulation is administered subcutaneously or by parenteral injection. In embodiments, the pharmaceutical dosage is an aqueous formulation comprising one or more of polysorbate 20, arginine, or mannitol. In embodiments, the pharmaceutical dosage formulation comprises about 0.025-0.5% (w/w) polysorbate 20, about 0.2-0.5% (w/w) arginine, and about 3-6% (w/w) mannitol in water (pH 7.7±0.1). In embodiments, the pharmaceutical dosage formulation comprises about 0.2% (w/w) polysorbate 20, about 0.348% (w/w) arginine, about 4.260% (w/w) mannitol in water (pH 7.7±0.1). In embodiments, the weekly therapeutic effective amount of the pharmaceutical dosage formulation comprises about 1.2 mg, 1.8 mg or 2.4 mg of the peptide product.

In embodiments, the pharmaceutical dosage comprises (2.4 mg/ml; 0.24% (w/w)) of the peptide product; Mannitol (42.6 mg/mL; 4.26% (w/w)); Arginine (3.48 mg/mL; 0.348% (w/w)); and polysorbate 20 (2 mg/mL; 0.2% (w/w)) in sterile water; (1.8 mg/ml; 0.18% (w/w)) of the peptide product; Mannitol (42.6 mg/mL; 4.26% (w/w)); Arginine (3.48 mg/mL; 0.348% (w/w)); and polysorbate 20 (2 mg/mL; 0.2% (w/w)) in sterile water; or, (1.2 mg/ml; 0.12% (w/w)) of the peptide product; Mannitol (42.6 mg/mL; 4.26% (w/w)); Arginine (3.48 mg/mL; 0.348% (w/w)); and polysorbate 20 (2 mg/mL; 0.2% (w/w)) in sterile water.

In certain embodiments, the methods of this disclosure further comprise administering at least one additional therapeutic intervention selected from the group consisting of: (a) anti-craving medications (such as naltrexone, acamprosate, or disulfiram), (b) antidepressants, (c) anxiolytics, and/or (d) behavioral therapy.

Other aspects of this disclosure are also contemplated as will be understood from the same by those of ordinary skill in the art.

### BRIEF DESCRIPTION OF THE DRAWINGS

FIG. 1 illustrates the increase in 15% ethanol consumption and reduction in tap water consumption over a 4-week induction period when the golden Syrian hamsters have free choice between normal tap water and 15% (v/v) ethanol in tap water (n=24). **p<0.01, ***p<0.001, ****p<0.0001, vs vehicle, with an ANOVA 2-way+Bonferonni's post-test. See Example 2.

FIG. 2 illustrates the effect of 10 nmol/kg pemvidutide treatment on mean body weight gain of golden Syrian hamsters over a 3-week treatment period as compared to vehicle control (n=12 for each group). ****p<0.0001, vs vehicle, with an ANOVA 2-way+Bonferonni's post-test. See Example 2

FIG. 3 illustrates the effect of 10 nmol/kg pemvidutide treatment on chow diet intake of golden Syrian hamsters (g/animal/day) over a 3-week treatment period as compared to vehicle control (n=12 for each group). *p<0.05, **p<0.01, ***p<0.001, ****p<0.0001, vs vehicle, with an ANOVA 2-way+Bonferonni's post-test. See Example 2.

FIG. 4A illustrates the effect of 10 nmol/kg pemvidutide treatment on liver weight (g) of golden Syrian hamsters as compared to vehicle control at the end of the 3-week treatment period (n=12 for each group). ****p<0.0001, vs vehicle, with a t-test.

FIG. 4B illustrates the effect of 10 nmol/kg pemvidutide treatment on relative liver weight (% body weight) of golden Syrian hamsters as compared to vehicle control at the end of the 3 week treatment period (n=12 for each group). ****p<0.0001, vs vehicle, with a t-test.

FIG. 5 illustrates the effect of 10 nmol/kg pemvidutide treatment on the consumption of tap water and 15% ethanol by golden Syrian hamsters over the 3-week treatment period wherein 15% ethanol consumption was reduced at the end of the treatment period and as compared to controls (n=12 for each group). ****p<0.0001, vs vehicle, with an ANOVA 2-way+Bonferonni's post-test.

FIG. 6 illustrates the effect of 10 nmol/kg pemvidutide treatment on the percent of 15% alcohol intake per day (alcohol/total volume) by golden Syrian hamsters over the 3-week treatment period (n=12 for each group). Results show that pemvidutide treatment reduced ethanol preference within just 2 days of treatment. ***p=0.0001, vs vehicle, and ****p<0.0001, vs vehicle, with unpaired T-test.

### DETAILED DESCRIPTION OF THE DISCLOSURE

This disclosure relates to methods for the treatment of Alcohol Use Disorder (AUD) and/or alcohol dependence, including preventing and/or treating AUD and/or alcohol dependence, in a human being in need thereof comprising administering to the human being a once weekly therapeutic effective amount of a pharmaceutical dosage formulation comprising a peptide or peptide product of this disclosure. AUD, alcohol dependence and alcohol misuse are used interchangeably herein. In embodiments, the human being in need thereof is obese or overweight, or has one or more co-morbidities such as fatty liver disease, type 2 diabetes, polycystic ovary syndrome (PCOS), cardiovascular disease, hypertension, dyslipidemia, hyperglycemia, or hyperinsulinemia. In certain embodiments, the human being exhibits conditions driven primarily by alcohol misuse such as alcohol-related liver disease (ARLD or ALD) including alcoholic fatty liver disease (AFLD), alcoholic steatohepatitis (ASH), metabolic dysfunction-associated steatotic liver disease (MetALD), alcohol-associated acute hepatitis (AH) or alcohol-associated cirrhosis (AC). In certain embodiments, the treatment methods disclosed herein reduce a symptom of withdrawal or prevent a relapse of the AUD, reduce tolerance to alcohol, reduce dependence on alcohol, improve adherence to a treatment for alcohol abuse, reduce a preference for alcohol, decrease liking for alcohol, or increase abstinence from alcohol.

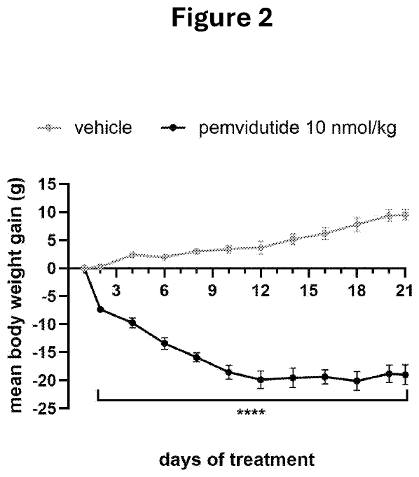

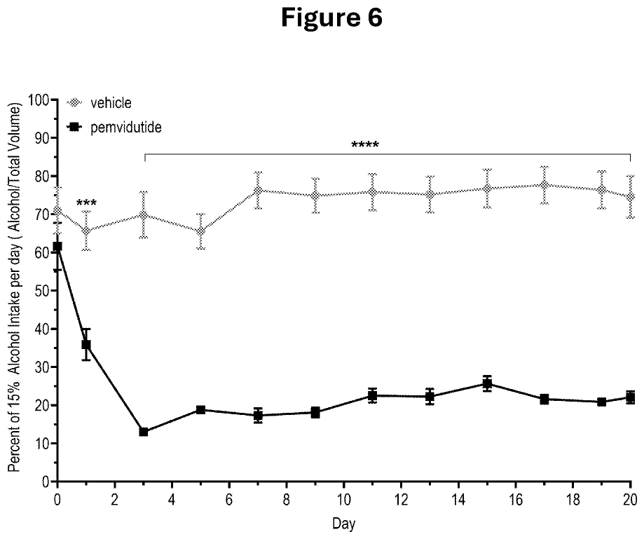

The applicant herein demonstrates that a peptide product of this disclosure (e.g., SEQ ID NO: 1) reduces alcohol (i.e., ethanol) consumption, and preference thereof, in a chronic alcohol intake animal model, during a treatment period of 3 weeks. Surprisingly, treatment with SEQ ID NO: 1 reduces the preference for alcohol consumption within just two days and the effect persisted through the end of the treatment period. See FIG. 6. In addition, SEQ ID NO: 1 treated hamsters also had significant reductions in food intake, body weight and liver weight compared to vehicle. See FIGS. 2-5. Accordingly, provided herein are methods for the treatment of AUD using peptide or peptide products of this disclosure, wherein the methods decrease ethanol-craving and/or ethanol use in humans suffering from Alcohol Use Disorder (AUD) (i.e., “a human being in need thereof”). See Example 2 and 3. Reduction in alcohol consumption can be measured through self-reporting, measuring reduction of phosphatidylethanol (PEth) levels, and/or measuring reduction of liver enzymes such as alanine aminotransferase (ALT) and/or aspartate aminotransferase (AST). PEth is used for detecting unhealthy alcohol consumption (e.g., such as binge drinkers, and/or persons with moderate drinking habits (i.e., exceeding 20 g of pure ethanol per day)) in an individual and quantified in blood through liquid chromatography coupled to mass spectrometry, wherein a correlation between blood PEth level and the amount of alcohol ingested in the previous two weeks is generally observed.

PEth represents a group of anomalous negatively-charged diacyl phospholipids formed in different human cells in the presence of ethanol. These lipids are derived in vivo from phosphatidylcholines via transphosphatidylation reaction catalyzed by phospholipase D only in the presence of ethanol. PEth formation occurs in different cells and tissues, such as erythrocytes, platelets, lymphocytes, brain, and liver, but PEth lipids accumulate only in red blood cells due to the inactivity of phospholipase C. Compared to other biomarkers, PEth quantification can detect even low levels of alcohol consumption over a longer time window, since they are detectable for about three to four weeks of daily intake of 50 g ethanol and up to approximately two weeks after ceasing alcohol intake. The most common analytical technique employed to quantify PEth in blood is liquid chromatography coupled to mass spectrometry (LC-MS or LC-MS/MS), due to its high sensitivity and ability to distinguish between different PEth molecular species. Peth subtypes may vary in abundance and specificity. PEth 16:0/18:1 (Palmitoyl-Oleoyl PEth) is the most prevalent in blood and is strongly associated with chronic and excessive alcohol consumption, making it a key marker in clinical evaluations. PEth 16:0/18:2 (Palmitoyl-Linoleoyl PEth), the second most common subtype, is also linked to moderate-to-heavy drinking and is often measured alongside PEth 16:0/18:1 to enhance diagnostic accuracy. PEth 18:1/18:1 (Dioleoyl PEth) is less abundant but remains a relevant indicator of alcohol exposure, particularly in chronic users, and may reflect recent intake due to its shorter elimination half-life. Similarly, PEth 18:0/18:2 (Stearoyl-Linoleoyl PEth) is less frequently detected but contributes to confirming alcohol consumption patterns when analyzed with other PEth subtypes. Lastly, PEth 16:0/16:0 (Diplamitoyl PEth) is present at lower levels and may provide additional insights into PEth metabolism and alcohol use.

Alanine aminotransferase (ALT) and aspartate aminotransferase (AST) are enzymes found in the liver and when the liver is damaged or inflamed ALT and/or AST are released into the bloodstream. Measuring the level of those enzymes in blood is a biomarker for liver damage. Provided herein are methods for the treatment of AUD that reduce blood levels of ALT and/or AST.

Other biomarkers for alcohol consumption include carbohydrate-deficient transferrin (CDT), a glycoprotein variant whose levels increase during chronic heavy alcohol intake, Additional biomarkers of interest comprise ethyl glucuronide (EtG) and ethyl sulfate (EtS)—metabolites typically identifiable in bodily fluids for up to several days post-consumption—and gamma-glutamyl transferase (GGT), an enzyme that may be elevated by prolonged alcohol use, although it can also rise in other liver conditions. Markers such as mean corpuscular volume (MCV) can further indicate harmful alcohol use. While some of these markers (e.g., GGT, AST, ALT, MCV) may be less specific due to elevations in other liver or hematologic disorders, their combined evaluation—especially with more specific indicators such as PEth or CDT—can reliably characterize a patient's recent and long-term alcohol consumption patterns.

In addition, the two-stage reduction in World Health Organization (WHO) drinking levels may also serve as a clinically relevant measure for evaluating alcohol consumption in clinical trials, providing a structured framework for assessing reductions in drinking-related health risks. In accordance with the WHO guidelines, this approach involves an initial decrease of at least one drinking risk level, followed by a further reduction to a lower-risk category or abstinence.

Non-invasive assessment methods to monitor the effect of alcohol intake on liver function may include various biomarkers such as cytokines, immune activation markers, endotoxemia indicators, and imaging modalities, providing valuable insights into disease progression and severity. Pro-inflammatory cytokines such as high-sensitivity C-reactive protein (hsCRP), tumor necrosis factor-alpha (TNF-α), interleukin-6 (IL-6), and interleukin-1 beta (IL-1β) indicate hepatocellular injury and fibrosis progression, while IL-17, IL-18, IL-22, IL-23, and TWEAK contribute to immune activation, hepatocyte injury, and fibrosis progression. Markers of innate and adaptive immune activation, including myeloperoxidase (MPO), neutrophil gelatinase-associated lipocalin (NGAL), soluble intercellular adhesion molecule-1 (sICAM-1), soluble vascular cell adhesion molecule-1 (sVCAM-1), and galectin-3, reflect neutrophil activity, endothelial dysfunction, and macrophage-driven inflammation. Anti-inflammatory cytokines like IL-10 modulate immune responses but may be dysregulated in chronic liver disease, leading to persistent inflammation, while fibrogenic cytokines such as transforming growth factor-beta (TGF-β) and platelet-derived growth factor (PDGF) promote hepatic stellate cell activation and extracellular matrix deposition, contributing to fibrosis progression. Chemokines such as C—C motif chemokine ligand 2 (CCL2/MCP-1) drive monocyte recruitment and fibrosis, whereas monocyte and macrophage activation markers like MCP-3, macrophage migration inhibitory factor (MIF), and soluble triggering receptor expressed on myeloid cells-1 (sTREM-1) highlight immune cell recruitment and systemic inflammatory responses. Endothelial dysfunction and coagulation markers, including von Willebrand Factor (vWF), plasminogen activator inhibitor-1 (PAI-1), and D-dimer, indicate vascular involvement in ALD pathology, while microbiome-related inflammatory markers such as zonulin, short-chain fatty acids (SCFAs), and indoleamine 2,3-dioxygenase (IDO) underscore the gut-liver axis's role in disease progression. Markers of endotoxemia and gut barrier dysfunction, including the Endotoxin Activity Assay (EAA), Limulus Amebocyte Lysate (LAL) assay, total bacterial 16S rDNA, soluble CD14 (sCD14), soluble CD163 (sCD163), and LPS-binding protein (LBP), offer insights into bacterial translocation and immune activation. Oxidative stress markers such as F2-isoprostanes assess lipid peroxidation and oxidative damage, contributing to hepatocyte injury and fibrosis. Fibrosis progression can be monitored through biomarkers like the Enhanced Liver Fibrosis (ELF) score, hyaluronic acid (HA), procollagen III N-terminal peptide (P3NP), tissue inhibitor of metalloproteinases-1 (TIMP-1), cytokeratin-18 (CK18) fragments M30/M65, and Mac-2 Binding Protein Glycosylation Isomer (M2BPGi), with ELF integrating HA, P3NP, and TIMP-1 to provide a quantitative measure of fibrosis severity. Additional fibrosis markers include proteins from genes such as IGLV3-21, IGHV1OR15-1, TFRC, IGKV1-27, KRT18, THBS4, IGKV2-40, IGHV2-26, FGA, IGKV6D-21, CD44, ACTA2, C1QB, CST3, IGHV3-38, FGG, ECM1, SELL, PROCR, IGHV3-72, CFP, PRDX1, PLG, C8B, CPN2, VTN, GALNT5, APOB, CPB2, WFDC3, CPN1, PCYOX1, CAMP, IGFALS, and C4A. Composite scoring systems such as ASH FibroSure, NASH-FibroTest, Somasignal, and MASEF Score integrate multiple biochemical markers to assess fibrosis, inflammation, and steatosis, while imaging modalities like Vibration Controlled Transient Elastography (VCTE) (FibroScan®), shear wave elastography (SWE), corrected T1 (cT1) imaging, Magnetic Resonance Elastography (MRE), and multiparametric MRI (proton density fat fraction (PDFF) and MR spectroscopy) provide non-invasive quantification of liver stiffness, fibrosis, inflammation, and hepatic fat.

This disclosure relates to pemvidutide (a preferred “dual agonist peptide”) as well as pharmaceutical dosage formulations comprising, and methods for using, the same. Pemvidutide, as a dual agonist peptide, has affinity for, and in preferred embodiments about equal affinity for, glucagon-like peptide 1 receptor (GLP-1R) and glucagon receptor (GCGR), as may be determined using a cellular assay. In preferred embodiments, this disclosure provides methods for using pemvidutide (e.g., preferably pharmaceutical dosage formulation comprising SEQ ID NO: 1) that upon administration to a human being reduces alcohol consumption and or maintains reduced consumption of alcohol in the human being. In certain embodiments, the human being is overweight, obese (e.g., a body mass index of at least about 25, with a range from at least 25 to at least 40). In certain embodiments, the human being in need thereof has a co-morbidity selected from one or more of alcohol associated liver disease, type 2 diabetes, polycystic ovary syndrome (PCOS), cardiovascular disease, hyperglycemia, and hyperinsulinemia. In certain embodiments, the administration of pemvidutide disclosed herein for treatment of AUD reduces excess body weight and/or treats fatty liver disease. In some embodiments, the disclosure provides pharmaceutical dosage formulations configured as a once weekly therapeutic effective amount of a pharmaceutical dosage formulation to reduce alcohol consumption and/or maintain reduced alcohol consumption in treatment of alcohol use disorder (AUD). In certain embodiments, the pharmaceutical dosage formulations comprising pemvidutide are administered in a once weekly therapeutically effective dose to reduce alcohol consumption and/or maintain reduced alcohol consumption.

Glucagon-like peptide-1 receptor agonists (GLP-1 RAs) containing compound such as pemvidutide may be also administered to treat a variety of addictive behaviors in addition to alcohol use disorder (AUD), including food addiction, tobacco smoking, cannabis dependence, cocaine use, and opioid abuse. Such agents may act, at least in part, by modulating the dopamine reward pathway, thereby reducing craving and drug-seeking behavior. Further, central GABA (gamma-aminobutyric acid) release and mitigation of negative emotional stress associated with abstinence or restriction (whether from food or other substances) may also contribute to the therapeutic effects of GLP-1-based compounds in substance use disorders. Accordingly, these compounds may provide a broad-spectrum approach to the management of multiple addictions, addressing neurobiological pathways shared by excessive alcohol intake, overeating, and illicit substance abuse, and thereby potentially improving outcomes across a range of dependence-related conditions.

In embodiments, the pharmaceutical dosage formulation disclosed herein comprises SEQ ID NO: 1, about 0.025-0.5% (w/w) polysorbate 20, about 0.2-0.5% (w/w) arginine, and about 3-6% (w/w) mannitol in water (pH 7.7±0.1). In exemplary embodiments, the pharmaceutical dosage formulation disclosed herein comprises SEQ ID NO: 1, about 0.2% (w/w) polysorbate 20, about 0.348% (w/w) arginine, about 4.260% (w/w) mannitol in water (pH 7.7±0.1).

As used herein, the terms “treat,” “treated,” “treatment” or “treating” mean both therapeutic treatment and prophylactic measures wherein the object is to slow down (lessen) an undesired physiological condition, disorder or disease, or obtain beneficial or desired clinical results. Beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response without excessive levels of side effects. Treatment also includes prolonging survival as compared to expected survival if not receiving treatment. Thus, “treatment of AUD” means an activity that alleviates or ameliorates any of the primary phenomena or secondary symptoms associated with the alcohol consumption, alcohol dependence or other conditions described herein, including, but not limited to alcohol fatty liver disease (AFDL), type 2 diabetes, polycystic ovary syndrome (PCOS), cardiovascular disease, hyperglycemia, and hyperinsulinemia.

In certain embodiments provided herein are methods for treatment of AUD, (e.g., reducing alcohol consumption or preventing relapse) comprises administering to a human subject in need thereof a once weekly therapeutically effective dose of at least 1.2 mg and up to 2.4 mg of pemvidutide for one or more weeks. Pemvidutide, present as a pharmaceutical formulation and comprising a synthetic peptide product (SEQ ID NO: 1) is composed of naturally occurring amino acids and is a chimeric analog of the two native hormones GLP-1 and glucagon, with predominantly glucagon residues in the N-terminus and GLP-1 residues in the C-terminus. See U.S. Pat. No. 9,856,306, incorporated herein by reference. Pemvidutide also incorporates one nonproteinogenic amino acid, 2-aminoisobutyric acid, an amino acid side chain amide linkage (lactam bridge), and a surfactant side chain composed of a glucuronic acid linked to an octadecanoic fatty acid side chain. The surfactant side chain appears to slow entry in the circulation and may form micelles after subcutaneous (SC) injection. The lower maximal concentration (Cₘₐₓ) associated with slower entry may result in fewer GI side effects and better tolerability. This latter feature also enhances binding to plasma proteins and improves the metabolic stability, extending the half-life (t_{1/2}). The design of pemvidutide provides a co-agonist with equipotent (1:1) activity at both receptors of approximately 40 μM and 100% activity. Compositions comprising pemvidutide have been administered to human beings across a variety of doses and found not to induce side effects such as nausea, vomiting, diarrhea, abdominal pain and/or constipation using standard techniques. See US Patent Publ. No. 2021/0290732 and PCT Publication No. WO 2022/125598, each incorporated herein by reference.

SEQ ID NO: 1 has the following amino acid sequence:
¹His-²Aib-³Gln-⁴Gly-⁵Thr-⁶Phe-⁷Thr-⁸Ser-⁹Asp-¹⁰Tyr-¹¹Ser-¹²Lys-¹³Tyr-¹⁴Leu-¹⁵Asp-¹⁶Glu*-¹⁷Lys^{#}-¹⁸Ala-¹⁹Ala-²⁰Lys*_²¹Glu-²²Phe-²³Ile-²⁴Gln-²⁵Trp-²⁶Leu-²⁷Leu-²⁸Gln-²⁹Thr-NH₂,
   where * indicates a lactam bridge is formed between ¹⁶Glu and ²⁰Lys, and ¹⁷Lys^{#} indicates the attachment site for glucuronic acid C-18-COOH) (“1-(17-carboxylheptadecyloxy)-beta-D-glucuronyl”). Illustrated differently, SEQ ID NO: 1 is a peptide amide consisting of 29 amino acid residues and a glucuronic acid/C18 diacid moiety attached to ¹⁷Lys, in which the side-chains of ¹⁶Glu and ²⁰Lys form an intramolecular cycle as shown below:

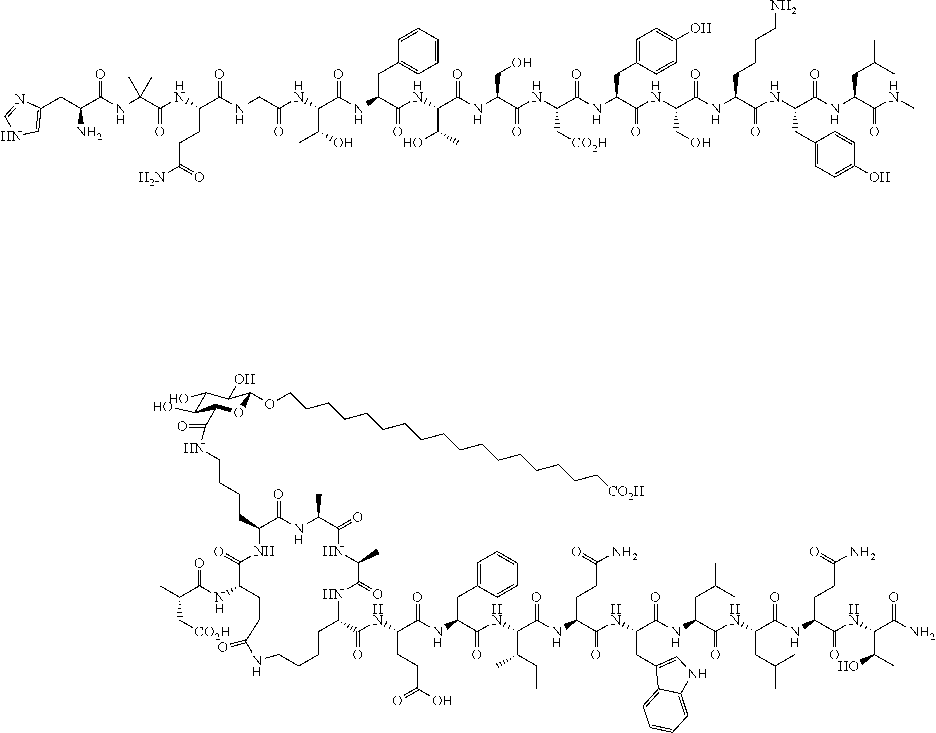

In embodiments provided herein is a pharmaceutical formulation of SEQ ID NO: 1 (referred to herein as pemvidutide) in an aqueous buffer solution. Pemvidutide comprises an amino acid side chain amide linkage (lactam bridge), and a surfactant side chain composed of a glucuronic acid linked to a fatty acid side chain. The side chain, a surfactant comprised of a hydrophilic saccharide group covalently attached to the peptide via a linker amino acid, and a hydrophobic alkyl chain portion. The synthesis of SEQ ID NO. 1 is described in Example 1. In some embodiments, the dual agonist peptides can include one or more conservatively substituted amino acids as described herein. In certain embodiments, SEQ ID NO: 1 can include one or more conservatively substituted amino acids, but preferably not at amino acid residues 16, 17, or 20 to provide a pemvidutide derivative. Provided herein is a peptide product that is 100% identical to SEQ ID NO: 1. In alternative embodiments, the peptide product is at least 85%, at least 90%, at least 95%, at least 96%, at least 97%, at least 98% or at least 99% identical to a peptide of SEQ ID NO: 1. In embodiments, the peptide product is at least 90% identical, at least 91% identical, at least 92% identical, at least 93% identical, at least 94% identical, at least 95% identical, at least 96%, at least 97%, at least 98% or at least 99% identical to a peptide of SEQ ID NO: 1. In embodiments, provided herein are methods wherein a peptide product that is at least 95% identical to SEQ ID NO: 1 is administered to a human being in need thereof in a pharmaceutical formulation of this disclosure.

A “peptide” (e.g., dual agonist peptide) comprises two or more natural or/and unnatural amino acid residues linked typically via peptide bonds. Such amino acids can include naturally occurring structural variants, naturally occurring non-proteinogenic amino acids, or/and synthetic non-naturally occurring analogs of natural amino acids. The terms “peptide” and “polypeptide” are used interchangeably herein. Peptides include short peptides (about 2-20 amino acids), medium-length peptides (about 21-50 amino acids) and long peptides (> about 50 amino acids, which can also be called “proteins”). In some embodiments, a peptide product comprises a surfactant moiety covalently and stably attached to a peptide of no more than about 50, 40 or 30 amino acids. Synthetic peptides can be synthesized using an automated peptide synthesizer, for example. Peptides can also be produced recombinantly in cells expressing nucleic acid sequences that encode the peptides. Conventional notation is used herein to portray peptide sequences: the left-hand end of a peptide sequence is the amino (N)-terminus, and the right-hand end of a peptide sequence is the carboxyl (C)-terminus. Standard one-letter and three-letter abbreviations for the common amino acids are used herein. Although the abbreviations used in the amino acid sequences disclosed herein represent L-amino acids unless otherwise designated as D- or DL- or the amino acid is achiral, the counterpart D-isomer generally can be used at any position (e.g., to resist proteolytic degradation). Abbreviations for other amino acids used herein include: Aib=α-aminoisobutyric acid (or 2-methylalanine or Ca-methylalanine); Xaa: any amino acid, typically specifically defined within a formula. Abbreviations for other amino acids that can be used as described herein include: Ac3c=1-aminocyclopropane-1-carboxylic acid; Ac4c=1-aminocyclobutane-1-carboxylic acid; Ac5c=1-aminocyclopentane-1-carboxylic acid; Ac6c=1-aminocyclohexane-1-carboxylic acid; Aib=alpha-aminoisobutyric acid (or 2-methylalanine or Calpha-methylalanine); Bip=3-(biphenyl-4-yl) alanine; Bip2Et=3-(2′-ethylbiphenyl-4-yl) alanine; Bip2EtMeO=3-(2′-ethyl-4′-methoxybiphenyl-4-yl) alanine; Cit=citrulline; Deg=2,2-diethylglycine; Dmt=(2,6-dimethyl) tyrosine; 2FPhe=(2-fluorophenyl) alanine; 2FMePhe or 2FaMePhe=Ca-methyl-(2-fluorophenyl) alanine; hArg=homoarginine; MeLys or aMeLys=Ca-methyllysine; MePhe or aMePhe=Ca-methylphenylalanine; MePro or aMePro=Ca-methylproline; Nall or Nal(l)=3-(l-naphthyl) alanine; Nal2 or Nal(2)=3-(2-naphthyl) alanine; Nle=norleucine; Om=ornithine; and Tmp=(2,4,6-trimethylphenyl) alanine; 1,2,3,4-tetrahydroisoquinoline-3-carboxylic acid (Tic) and a Tic-Phe dipeptide moiety with a reduced amide bond between the residues (designated as Tic-Ψ[CFl2-NFl]-Ψ-Phe) have the following structures:

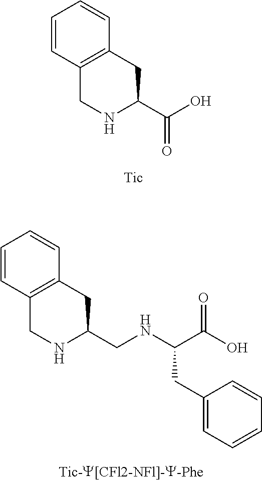 [SMILES: O=C(O)[C@@H]1Cc2ccccc2CN1 ; O=C(O)[C@H](Cc1ccccc1)NC[C@@H]1Cc2ccccc2CN1]

Unless specifically stated otherwise or the context clearly indicates otherwise, the disclosure encompasses any and all forms of a dual agonist peptide that may be produced, whether the dual agonist peptide is produced synthetically (e.g., using a peptide synthesizer) or by a cell (e.g., by recombinant production). Such forms of a dual agonist peptide can include one or more modifications that may be made during the course of synthetic or cellular production of the peptide, such as one or more post-translational modifications, whether or not the one or more modifications are deliberate. A dual agonist peptide can have the same type of modification at two or more different places, or/and can have two or more different types of modifications. Modifications that may be made during the course of synthetic or cellular production of a dual agonist peptide, including chemical and post-translational modifications, include without limitation glycosylation (e.g., N-linked glycosylation and O-linked glycosylation), lipidation, phosphorylation, sulfation, acetylation (e.g., acetylation of the N-terminus), amidation (e.g., amidation of the C-terminus), hydroxylation, methylation, formation of an intramolecular or intermolecular disulfide bond, formation of a lactam between two side chains, formation of pyroglutamate, and ubiquitination. A dual agonist peptide can have one or more modifications anywhere, such as the N-terminus, the C-terminus, one or more amino acid side chains, or the dual agonist peptide backbone, or any combination thereof. In some embodiments, a dual agonist peptide is acetylated at the N-terminus or/and has a carboxamide (—CONH₂) group at the C-terminus, which can increase the stability of the dual agonist peptide.

Potential modifications of pemvidutide can also include deletion of one or more amino acids, addition/insertion of one or more natural or/and unnatural amino acids, or substitution with one or more natural or/and unnatural amino acids, or any combination or all thereof. A substitution can be conservative or non-conservative. Such modifications may be deliberate, such as via site-directed mutagenesis or in the chemical synthesis of a dual agonist peptide, or may be accidental, such as via mutations arising in the host cell that produces the dual agonist peptide or via errors due to PCR amplification. An unnatural amino acid can have the same chemical structure as the counterpart natural amino acid but have the D stereochemistry, or it can have a different chemical structure and the D or L stereochemistry. Unnatural amino acids can be utilized, e.g., to promote a-helix formation or/and to increase the stability of the dual agonist peptide (e.g., to resist proteolytic degradation). A dual agonist peptide having one or more modifications relative to a reference dual agonist peptide may be called an “analog” or “variant” of the reference dual agonist peptide as appropriate. An “analog” typically retains one or more essential properties (e.g., receptor binding, activation of a receptor or enzyme, inhibition of a receptor or enzyme, or other biological activity) of the reference dual agonist peptide. A “variant” may or may not retain the biological activity of the reference dual agonist peptide, or/and may have a different biological activity. It is preferred that such a variant maintains its ability to act as an agonist of GLP-1R and GCGR, and in more preferred embodiments, has about equal affinity for GLP-1R and GCGR. In some embodiments, an analog or variant of a reference peptide has a different amino acid sequence than the reference dual agonist peptide.

The term “conservative substitution” refers to substitution of an amino acid in a dual agonist peptide with a functionally, structurally or chemically similar natural or unnatural amino acid. In certain embodiments, the following groups each contain natural amino acids that are conservative substitutions for one another: 1) Glycine (Gly/G), Alanine (Ala/A); 2) Isoleucine (Ile/I), Leucine (Leu/L), Methionine (Met/M), Valine (Val/V); 3) Phenylalanine (Phe/F), Tyrosine (Tyr/Y), Tryptophan (Trp/W); 4) Serine (Ser/S), Threonine (Thr/T), Cysteine (Cys/C); 5) Asparagine (Asn/N), Glutamine (Gln/Q); 6) Aspartic acid (Asp/D), Glutamic acid (Glu/E); and, 7) Arginine (Arg/R), Lysine (Lys/K), Histidine (His/H). In further embodiments, the following groups each contain natural amino acids that are conservative substitutions for one another: 1) non-polar: Ala, Val, Leu, Ile, Met, Pro (proline/P), Phe, Trp; 2) hydrophobic: Val, Leu, Ile, Phe, Trp; 3) aliphatic: Ala, Val, Leu, Ile; 4) aromatic: Phe, Tyr, Trp, His; 5) uncharged polar or hydrophilic: Gly, Ala, Pro, Ser, Thr, Cys, Asn, Gln, Tyr; 6) aliphatic hydroxyl- or sulfhydryl-containing: Ser, Thr, Cys; 7) amide-containing: Asn, Gln; 8) acidic: Asp, Glu; 9) basic: Lys, Arg, His; and, 10) small: Gly, Ala, Ser, Cys. In other embodiments, amino acids may be grouped as conservative substitutions as set out below: 1) hydrophobic: Val, Leu, Ile, Met, Phe, Trp; 2) aromatic: Phe, Tyr, Trp, His; 3) neutral hydrophilic: Gly, Ala, Pro, Ser, Thr, Cys, Asn, Gln; 4) acidic: Asp, Glu; 5) basic: Lys, Arg, His; and, 6) residues that influence backbone orientation: Pro.

Examples of unnatural or non-proteinogenic amino acids include without limitation alanine analogs (e.g., α-ethylGly [α-aminobutyric acid or Abu], α-n-propylGly [norvaline or Nva], α-tert-butylGly [Tbg], α-vinyl Gly [Vg or Vig], α-allylGly [Alg], α-propargylGly [Prg], 3-cyclopropylAla [Cpa] and Aib), leucine analogs (e.g., nor-leucine, Nle), proline analogs (e.g., α-MePro), phenylalanine analogs (e.g., Phe (2-F), Phe (2-Me), Tmp, Bip, Bip (2′-Et-4′-OMe), Nal1, Nal2, Tic, α-MePhe, α-MePhe (2-F) and α-MePhe (2-Me)), tyrosine analogs (e.g., Dmt and α-MeTyr), serine analogs (e.g., homoserine [isothreonine or hSer]), glutamine analogs (e.g., Cit), arginine analogs (e.g., hArg, N,N′-g-dialkyl-hArg), lysine analogs (e.g, homolysine [hLys], Orn and α-MeLys), α,α-disubstituted amino acids (e.g., Aib, α,α-diethylGly [Deg], α-cyclohexylAla [2-Cha], Ac3c, Ac4c, Ac5c and Ac6c), and other unnatural amino acids disclosed in A. Santoprete et al., Pept. Sci., 17:270-280 (2011). α,α-Di-substituted amino acids can provide conformational restraint or/and α-helix stabilization. A reduced amide bond between two residues (as in, e.g., Tic-Ψ[CFl2-NFl]-Ψ-Phe) increases protease resistance and may also, e.g., alter receptor binding. The disclosure encompasses all pharmaceutically acceptable salts of dual agonist peptides, including those with a positive net charge, those with a negative net charge, and those with no net charge.

In some embodiments, for instance, the dual agonist peptides can be associated with a saccharide, such as within a pharmaceutically acceptable composition or lyophilizate. Saccharides include monosaccharides, disaccharides and oligosaccharides (e.g., trisaccharides, tetrasaccharides and so on). A reducing saccharide exists in a ring form and an open-chain form in equilibrium, which generally favors the ring form. A functionalized saccharide of a surfactant moiety has a functional group suitable for forming a stable covalent bond with an amino acid of a dual agonist peptide.

The term “pharmaceutically acceptable” refers to a substance (e.g., an active ingredient or an excipient) that is suitable for use in contact with the tissues and organs of a subject without excessive irritation, allergic response, immunogenicity and toxicity, is commensurate with a reasonable benefit/risk ratio, and is effective for its intended use. A “pharmaceutically acceptable” excipient or carrier of a pharmaceutical composition is also compatible with the other ingredients of the composition. In one embodiment, a pharmaceutically acceptable composition in which pemvidutide (or a derivative thereof) can be formulated comprises polysorbate 20 (e.g., about 0.2% (w/w)); optionally methylparaben (e.g., about 0.300% (w/w)); arginine (about 0.348% (w/w)), and mannitol (e.g., about 4.260% (w/w)) in distilled (DI) water.

The term “therapeutically effective amount” refers to an amount of a compound that, when administered to a subject, is sufficient to prevent, reduce the risk of developing, delay the onset of, slow the progression of or cause regression of the medical condition being treated, or to alleviate to some extent the medical condition or one or more symptoms or complications of that condition, at least in some fraction of the subjects taking that compound. The term “therapeutically effective amount” also refers to an amount of a compound that is sufficient to elicit the biological or medical response of a cell, tissue, organ or human which is sought by a medical doctor or clinician.

The disclosure also provides pharmaceutical compositions comprising a dual agonist peptide product described herein or a pharmaceutically acceptable salt thereof, and one or more pharmaceutically acceptable carriers or excipients. A pharmaceutical composition contains a therapeutically effective amount of a peptide product or an appropriate fraction thereof. A composition can optionally contain an additional therapeutic agent. In some embodiments, a peptide product is at least about 90%, 95% or 98% pure. Pharmaceutically acceptable excipients and carriers include pharmaceutically acceptable substances, materials and vehicles. Non-limiting examples of types of excipients include liquid and solid fillers, diluents, binders, lubricants, glidants, surfactants, dispersing agents, disintegration agents, emulsifying agents, wetting agents, suspending agents, thickeners, solvents, isotonic agents, buffers, pH adjusters, absorption-delaying agents, stabilizers, antioxidants, preservatives, antimicrobial agents, antibacterial agents, antifungal agents, chelating agents, adjuvants, sweetening agents, flavoring agents, coloring agents, encapsulating materials and coating materials. The use of such excipients in pharmaceutical formulations is known in the art. For example, conventional vehicles and carriers include without limitation oils (e.g., vegetable oils such as olive oil and sesame oil), aqueous solvents (e.g., saline, buffered saline (e.g., phosphate-buffered saline [PBS]) and isotonic solutions (e.g., Ringer's solution)), and organic solvents (e.g., dimethyl sulfoxide and alcohols [e.g., ethanol, glycerol and propylene glycol]). Except insofar as any conventional excipient or carrier is incompatible with a peptide product, the disclosure encompasses the use of conventional excipients and carriers in formulations containing a peptide product. See, e.g., Remington: The Science and Practice of Pharmacy, 21st Ed., Lippincott Williams & Wilkins (Philadelphia, Pennsylvania) (2005); Handbook of Pharmaceutical Excipients, 5th Ed., Rowe et ah, Eds., The Pharmaceutical Press and the American Pharmaceutical Association (2005); Handbook of Pharmaceutical Additives, 3rd Ed., Ash and Ash, Eds., Gower Publishing Co. (2007); and Pharmaceutical Pre-formulation and Formulation, Gibson, Ed., CRC Press (Boca Raton, Florida) (2004).

In embodiments, a pharmaceutical formulation comprises a peptide product of this disclosure and about 0.025-0.5% (w/w) polysorbate 20, about 0.2-0.5% (w/w) arginine, about 3-6% (w/w) mannitol in water (e.g. deionized and/or sterilized) (pH 7.7±0.1); optionally about 0.2% (w/w) polysorbate 20, about 0.348% (w/w) arginine, about 4.260% (w/w) mannitol in water (pH 7.7±0.1). In certain embodiments, a present pharmaceutical formulation comprises SEQ ID NO: 1 (or a peptide product that is at least 80% identical to SEQ ID NO: 1) and about 0.2% (w/w) polysorbate 20, about 0.348% (w/w) arginine, about 4.260% (w/w) mannitol in water (pH 7.7±0.1). In certain embodiments, the pharmaceutical formulation comprises SEQ ID NO: 1 (or a peptide product that is at least 80% identical to SEQ ID NO: 1) and is configured for subcutaneous (SC) administration of a weekly therapeutic effective dose.

In certain embodiments, a peptide product of this disclosure is provided in a therapeutically effective dose of 1.2 mg/mL to 2.4 mg/mL. In certain embodiments the therapeutic formulation comprises peptide product (2.4 mg/ml; 0.24% (w/w)); Mannitol (42.6 mg/mL; 4.26% (w/w)); Arginine (3.48 mg/mL; 0.348% (w/w)); and polysorbate 20 (2 mg/mL; 0.2% (w/w)) in sterile water. In certain embodiments the therapeutic formulation comprises peptide product (1.8 mg/ml; 0.18% (w/w)); Mannitol (42.6 mg/mL; 4.26% (w/w)); Arginine (3.48 mg/mL; 0.348% (w/w)); and polysorbate 20 (2 mg/mL; 0.2% (w/w)) in sterile water. In certain embodiments the therapeutic formulation comprises peptide product (1.2 mg/ml; 0.12% (w/w)); Mannitol (42.6 mg/mL; 4.26% (w/w)); Arginine (3.48 mg/mL; 0.348% (w/w)); and polysorbate 20 (2 mg/mL; 0.2% (w/w)) in sterile water. In embodiments, the peptide product is pemvidutide. In embodiments, the peptide product is SEQ ID NO: 1. In certain embodiments, the peptide product is at least 80%, at least 85%, at least 90% or at least 95% identical to SEQ ID NO: 1.

An appropriate or suitable formulation can depend on various factors, such as the route of administration chosen. Potential routes of administration of a pharmaceutical composition comprising a peptide product include without limitation oral, parenteral (including intradermal, subcutaneous, intramuscular, intravascular, intravenous, intra-arterial, intraperitoneal, intracavitary and topical), and topical (including transdermal, transmucosal, intranasal (e.g., by nasal spray or drop), ocular (e.g., by eye drop), pulmonary (e.g., by oral or nasal inhalation), buccal, sublingual, rectal (e.g., by suppository), and vaginal (e.g., by suppository). In certain embodiments, a present dual agonist peptide product is administered parenterally (e.g., subcutaneously, intravenously or intramuscularly). In other embodiments, a peptide product is administered by oral inhalation or nasal inhalation or insufflation. In some embodiments, the carrier is an aqueous-based carrier, such as in a parenteral (e.g., subcutaneous, intravenous or intramuscular) formulation. In other embodiments, the carrier is a nonaqueous-based carrier. In certain embodiments, the nonaqueous-based carrier is a hydrofluoroalkane (HFA) or HFA-like solvent that may comprise sub-micron anhydrous α-lactose or/and other excipients, such as in a formulation for administration by oral inhalation or nasal inhalation or insufflation.

In some embodiments, a peptide product is administered parenterally (e.g., subcutaneously, intravenously or intramuscularly) by injection. Parenteral administration bypasses the strongly acidic environment of the stomach, gastrointestinal (GI) absorption and first-pass metabolism. Excipients and carriers that can be used to prepare parenteral formulations include without limitation solvents (e.g., aqueous solvents such as water, saline, physiological saline, buffered saline [e.g., PBS], balanced salt solutions [e.g., Ringer's BSS] and aqueous dextrose solutions), isotonic/iso-osmotic agents (e.g., salts [e.g., NaCl, KCl and CaCl₂)] and sugars [e.g., sucrose]), buffering agents and pH adjusters (e.g., sodium dihydrogen phosphate [monobasic sodium phosphate]/di sodium hydrogen phosphate [dibasic sodium phosphate], citric acid/sodium citrate and L-histidine/L-histidine HCl), and emulsifiers (e.g., non-ionic surfactants such as polysorbates [e.g., polysorbate 20 and 80] and poloxamers [e.g., poloxamer 188]). Peptide formulations and delivery systems are discussed in, e.g., A. J. Banga, Therapeutic Peptides and Proteins: Formulation, Processing, and Delivery Systems, 3rd Ed., CRC Press (Boca Raton, Florida) (2015). The excipients can optionally include one or more substances that increase peptide stability, increase peptide solubility, inhibit peptide aggregation or reduce solution viscosity, or any combination or all thereof. Such substances include without limitation hydrophilic amino acids (e.g., arginine and histidine), polyols (e.g., myo-inositol, mannitol and sorbitol), saccharides (e.g., glucose (including D-glucose [dextrose]), lactose, sucrose and trehalose}, osmolytes (e.g., trehalose, taurine, amino acids [e.g., glycine, sarcosine, alanine, proline, serine, b-alanine and g-aminobutyric acid], and betaines [e.g., trimethylglycine and trimethylamine N-oxide]), and non-ionic surfactants (e.g., alkyl polyglycosides, ProTek® alkylsaccarides (e.g., a monosaccharide [e.g., glucose] or a disaccharide [e.g., maltose or sucrose] coupled to a long-chain fatty acid or a corresponding long-chain alcohol), and polypropylene glycol/polyethylene glycol block co-polymers (e.g., poloxamers [e.g., Pluronic™F-68], and Genapol® PF-10 and variants thereof). Because such substances increase peptide solubility, they can be used to increase peptide concentration in a formulation. Higher peptide concentration in a formulation is particularly advantageous for subcutaneous administration, which has a limited volume of bolus administration (e.g., < about 1.5 mL). In addition, such substances can be used to stabilize peptides during the preparation, storage and reconstitution of lyophilized peptides. An exemplary parenteral formulation comprises a peptide product, mannitol, methionine, sodium thioglycolate, polysorbate 20, a pH adjuster (e.g., NaOH or/and HCl) and de-ionized water. Excipients of parenteral formulations that would be suitable for use with the dual agonist peptides described herein (e.g., various combinations of excipients including NaCl and the like) are well-known and available to those of ordinary skill in the art.

For parenteral (e.g., subcutaneous, intravenous or intramuscular) administration, a sterile solution or suspension of a peptide product in an aqueous solvent containing one or more excipients can be prepared beforehand and can be provided in, e.g., a pre-filled syringe of a single-use pen or a pen with a dose counter. Alternatively, a peptide product can be dissolved or suspended in an aqueous solvent that can optionally contain one or more excipients prior to lyophilization (freeze-drying). Shortly prior to parenteral administration, the lyophilized peptide product stored in a suitable container (e.g., a vial) can be reconstituted with, e.g., sterile water that can optionally contain one or more excipients.

A peptide product can also be administered by other routes, such as orally. An oral formulation can contain a peptide product and conventional excipients known in the art, and optionally an absorption enhancer such as sodium V-[8-(2-hydroxybenzoyl) aminocaprylate] (SNAC). SNAC protects against enzymatic degradation via local buffering action and enhances GI absorption. An oral dosage form (e.g., a tablet, capsule or pill) can optionally have an enteric coating to protect its content from the strong acids and proteolytic enzymes of the stomach. In some embodiments, a peptide product is delivered from a sustained-release composition. As used herein, the term “sustained-release composition” encompasses sustained-release, prolonged-release, extended-release, delayed-release, slow-release and controlled-release compositions, systems and devices. In some embodiments, a sustained-release composition delivers a peptide product over a period of at least about 1 week, 2 weeks, 3 weeks, 1 month, 2 months, 3 months or longer. In further embodiments, a sustained-release composition is in the form of a depot that is generated when a mixture of a peptide product and a polymer is injected into a subject intramuscularly or subcutaneously. In certain embodiments, the polymer is or comprises PEG, polylactic acid (PLA) or polyglycolic acid (PGA), or a copolymer thereof (e.g., PLGA or PLA-PEG).

A pharmaceutical composition can be presented in unit dosage form as a single dose wherein all active and inactive ingredients are combined in a suitable system, and components do not need to be mixed to form the composition to be administered. A unit dosage form generally contains a therapeutically effective dose of the drug but can contain an appropriate fraction thereof so that taking multiple unit dosage forms achieves the therapeutically effective dose. Examples of a unit dosage form include a tablet, capsule or pill for oral uptake; a solution in a pre-filled syringe of a single-use pen or a pen with a dose counter for parenteral (e.g., intravenous, subcutaneous or intramuscular) injection; and a capsule, cartridge or blister pre-loaded in or manually loaded into an inhaler. Alternatively, a pharmaceutical composition can be presented as a kit in which the active ingredient, excipients and carriers (e.g., solvents) are provided in two or more separate containers (e.g., ampules, vials, tubes, bottles or syringes) and need to be combined to form the composition to be administered. The kit can contain instructions for storing, preparing and administering the composition (e.g., a solution to be injected parenterally). A kit can contain all active and inactive ingredients in unit dosage form or the active ingredient and inactive ingredients in two or more separate containers, and can contain instructions for administering or using the pharmaceutical composition to treat a medical condition disclosed herein. A kit can further contain a device for delivering the composition, such as an injection pen or an inhaler. In some embodiments, a kit contains a peptide product or a pharmaceutically acceptable salt thereof, or a pharmaceutical composition comprising the same, and instructions for administering or using the peptide product or the composition to treat a medical condition disclosed herein, such Alcohol Use Disorder (AUD), or a condition associated therewith (e.g., obesity, type 2 diabetes or PCOS). In certain embodiments, the kit further contains a device for delivering the peptide product or the composition, such as an injection pen or an inhaler.

### Methods of Treatment

Provided herein are methods for the treatment of alcohol use disorder (AUD) and/or alcohol dependence comprising administering of a peptide product of this disclosure (e.g., SEQ ID NO: 1) to a subject in need thereof. In embodiments provided herein are methods for the treatment of alcohol use disorder (AUD) in a human being in need thereof comprising administering to the human being a once weekly therapeutic effective amount of a pharmaceutical dosage formulation comprising a peptide product according to SEQ ID NO: 1, or a peptide product that is at least 80% identical to a peptide of SEQ ID NO: 1. In embodiments, the treatment reduces a symptom of withdrawal or prevents a relapse of the alcohol use disorder in the human, reduces tolerance to alcohol, reduces dependence on alcohol, improves adherence to a treatment for alcohol abuse, reduces a preference for alcohol, decreases liking for alcohol, or increases abstinence from alcohol.

With dependence upon alcohol come a host of symptoms that work to perpetuate that dependence, and complicate curing or otherwise managing problematic substance use. For example, in many cases subjects addicted to alcohol develop an increased tolerance to alcohol. Tolerance frequently leads to increased consumption of alcohol to attempt to compensate for the diminished effects of the alcohol that can result from tolerance. Additional complicating symptoms include withdrawal symptoms that occur when alcohol consumption is stopped or reduced, cravings, reduced motivation for other reward stimuli, an increased sensitivity to stressful stimuli, a heightened sensitivity or responsivity to substance related cues, impulsivity, an inability to accurately evaluate delayed rewards, and a lack of motivation to change destructive behaviors, such as those caused by substance use.

In some embodiments, the symptoms that work to perpetuate alcohol use disorder in an individual result from changes in the brain that are caused by repeated alcohol consumption. Alternatively, or in addition, symptoms, such as impulsivity and stress sensitivity, may be present in an individual before the development of alcohol use disorder, and contribute to the susceptibility of that individual to alcohol use disorders. Genetic, environmental and psychiatric factors may all also contribute to an individual's vulnerability to developing alcohol use disorder.

In some embodiments, alcohol use disorders are connected to mood disorders, anxiety disorders or obsessive-compulsive disorder. For example, NIDA, the National Institute on Drug Abuse, indicates that subjects diagnosed with mood disorders are twice as likely to abuse substances as subjects without mood disorders. The combination of loss of reward function and recruitment of brain stress systems provides a powerful neurochemical basis for a negative emotional state that is responsible for the negative reinforcement driving, at least in part, the compulsivity of addiction. As use herein, “mood disorders” refer to refer to mental health disorders with a serious change in mood. Exemplary mood disorders include, but are not limited to major depressive disorder, bipolar disorder, dementia, low motivation, anxiety, insomnia, anorexia, obsessive compulsive disorder, post traumatic stress disorder, persistent depressive disorder, cyclothymia and seasonal affective disorder. Symptoms of mood disorders include, but are not limited to, anhedonia or negative affect, mood depression, low motivation, anxiety, insomnia, anorexia, impulsivity and stress sensitivity.

Multiple neurotransmitter systems have been implicated in alcohol use disorder, such as glutamate and Gamma-aminobutyric acid (GABA). In some embodiments, alcohol affects glutamate neurotransmitter signaling in the central nervous system of a subject with AUD. Glutamate is widely distributed in the brain. Exemplary glutamate functions comprise regulating the rate of neuronal activity (increasing the rate), learning, cognition and memory. In some embodiments, alcohol affects gamma aminobutyric acid (GABA) neurotransmitter signaling in the central nervous system of a subject with an alcohol use disorder. GABA is widely distributed in the brain. Exemplary GABA functions comprise regulating the rate of neuronal activity (decreasing the rate), anxiety, memory and anesthesia.

Changes in the brain that are linked to alcohol use disorder are referred to as adaptation or neuroadaptation, as the nervous system adapts to repeated use of the substance (i.e., alcohol). These changes may comprise changes in the expression or levels of neurotransmitter receptors, changes in the expression or levels of neurotransmitters, changes to neuronal structure, changes in neural connectivity or a combination thereof. Neuroadaptation is linked to the development of tolerance, wherein the reaction of an individual to a substance is reduced with repeated use, due in part to down regulation of receptors in the brain. Changes in these receptors may be involved in withdrawal symptoms as well as other symptoms associated with substance use disorders. Adaption to substance use is believed to contribute to many of the symptoms that make substance use disorders so difficult to treat. These symptoms include, but are limited to, tolerance, withdrawal, cravings, a heightened sensitivity to substance related cues, stress sensitivity, and low motivation to change destructive behaviors such as substance use, anxiety, irritability, difficulty concentrating, difficulty thinking clearly, mood swings, nightmares, depression, tension, panic attacks, short term memory loss, restlessness, a feeling of helplessness, stress-sensitivity, aberrant reward processing or a combination thereof.

Regions of the central nervous system that can be disrupted with alcohol use disorder and/or undergo adaptation caused by substance use include, but are not limited to, the midbrain, the ventral tegmental area (VTA), cerebral cortex, prefrontal cortex, hypothalamus, thalamus, hippocampus, basal ganglia, extended amygdala and cerebellum. Functions of the midbrain include, but are not limited to, contributing to motor control, visual and auditory processing, control of sleep/wake cycles, arousal and temperature regulation. The cerebral cortex is connected to various subcortical structures, such as the thalamus and basal ganglia. Functions of the cerebral cortex include, but are not limited to, contributing to motor and sensory processing, speech, memory, executive function, personality and social behavior. Functions of the prefrontal cortex, which is a part of the cerebral cortex, include, but are not limited to, contributing to higher cognitive functions such as anticipation, judgement, planning and decision making. Functions of the hypothalamus, include, but are not limited to, contributing to maintaining homeostasis of the autonomic nervous system, motivation and emotion. Functions of the thalamus include, but are not limited to, relaying sensory and motor signals and contributing to the regulation of consciousness, sleep and alertness. Functions of the hippocampus include, but are not limited to, regulating emotions and memory. Functions of the basal ganglia, include, but are not limited to, an involvement motor control, executive functions and behaviors, regulating mood, reward circuitry and learning. Functions of the extended amygdala include, but are not limited to, its role in processing emotions such as fear, stress, anxiety, irritability, anger and pleasure, as well as roles in motivation, arousal and memory. Functions of the cerebellum include, but are not limited to, receiving sensory information from the spinal cord and regulating motor movements, such as those involved in balance and coordination.

The symptoms of alcohol use disorder depend upon the duration and amount of use, and the subject. These symptoms can be physical or psychological in nature, or a combination thereof. Physical symptoms of substance use include, but are not limited to, tremors, insomnia, disturbed sleep, headache, sweating, nausea, vomiting, muscle pain, muscle stiffness, hypertension, irregular heart rate, elevated heart rate, heart palpitations, dizziness, shakiness, tremors, seizures, dehydration, shallow breathing, fatigue, loss of appetite, clammy skin, loss of color, metabolic disorders (e.g. type 2 diabetes, obesity and fatty liver disease) or a combination thereof. Psychological symptoms of alcohol use include, but are not limited to, anxiety, irritability, difficulty concentrating, difficulty thinking clearly, mood swings, nightmares, depression, tension, panic attacks, short term memory loss, restlessness, a feeling of helplessness, stress-sensitivity, a heightened responsivity to substance-related cues, aberrant reward processing, craving or a combination thereof.

Withdrawal is associated with alcohol use disorders of the disclosure. Withdrawal occurs when a subject accustomed to a relatively stable level of alcohol is suddenly deprived of alcohol. Withdrawal symptoms may be physical or psychological in nature, or a combination thereof. The specific withdrawal symptoms may depend on the amount and duration of alcohol use and the individual. Withdrawal symptoms may be immediate or delayed in onset. Physical symptoms of withdrawal include, but are not limited to, tremors, insomnia, disturbed sleep, headache, sweating, nausea, vomiting, muscle pain, muscle stiffness, hypertension, irregular heart rate, elevated heart rate, heart palpitations, dizziness, shakiness, tremors, seizures, dehydration, shallow breathing, fatigue, loss of appetite, clammy skin, loss of color or a combination thereof. Psychological symptoms of withdrawal include, but are not limited to, anxiety, irritability, difficulty concentrating, difficulty thinking clearly, mood swings, nightmares, depression, tension, panic attacks, short term memory loss, restlessness, a feeling of helplessness, stress-sensitivity, a heightened responsivity to substance-related cues, aberrant reward processing, craving or a combination thereof. In some embodiments, administering a therapeutically effective amount of a composition of this disclosure comprising a peptide product according to SEQ ID NO: 1, or a peptide product that is at least 80% identical to a peptide of SEQ ID NO: 1 reduces or prevents withdrawal symptoms from alcohol.

Tolerance is associated with alcohol use disorder of the disclosure. Tolerance occurs when an individual develops a reduced response to a substance with repeated use. The development of tolerance frequently leads to the individual consuming increasing amounts of alcohol, in an attempt to recreate the effect of the substance before tolerance developed. In some embodiments, administering a therapeutically effective amount of a composition comprising a peptide product according to SEQ ID NO: 1, or a peptide product that is at least 80% identical to a peptide of SEQ ID NO: 1 of the disclosure can decrease tolerance to alcohol. By blocking tolerance, methods of this disclosure reduce the amount of alcohol consumed. For example, reducing alcohol tolerance by administering a peptide product according to SEQ ID NO: 1, or a peptide product that is at least 80% identical to a peptide of SEQ ID NO: 1 would prevent drinkers from increasing the amount of alcohol consumed and thereby decrease the likelihood of developing dependence. In some embodiments, a peptide product according to SEQ ID NO: 1, or a peptide product that is at least 80% identical to a peptide of SEQ ID NO: 1 reduces the tolerance development to alcohol thereby reducing the risk of alcohol dependence.

Craving is a symptom associated with alcohol use disorder of the disclosure. Craving denotes a heightened desire for alcohol. Craving is often coupled to a difficulty in experiencing a cue, such as an environmental cue, without incurring a high level of desire for the substance. Craving can also include repeated failures to resist the urge to consume alcohol, and intolerable mental or physical states in the absence alcohol. Provided herein in certain embodiments, is a method for the treatment of AUD comprising administering a composition comprising a peptide product according to SEQ ID NO: 1, or a peptide product that is at least 80% identical to a peptide of SEQ ID NO: 1, to a human in need thereof wherein the treatment reduces cravings or preference for alcohol.

A heightened responsivity to alcohol related cues is a symptom associated with alcohol use disorders of the disclosure. Cue reactivity is a learned response, in which individuals with alcohol dependence develop physiological and/or subjective responses when presented with alcohol related stimuli. These stimuli can include, but are not limited to, bottles of alcohol (alcohol) or advertising. A heightened sensitivity to alcohol related cues can be a component of craving. Cue reactivity has been linked to increased activity in the amygdala, ventral striatum and prefrontal regions.

A heightened sensitivity to stress (“stress sensitivity”) is a symptom associated with alcohol use disorder of this disclosure. Stress is a well-known risk factor in the development of alcohol use disorder, and in the relapse of subjects who had previously managed to stop using a alcohol. Common stressors include early life (childhood) stress, economic stress and chronic or acute adverse life events. Stress is also associated with a variety of psychiatric disorders, such as mood and anxiety disorders, as well as post-traumatic stress disorder. Stress can lead to the release of hormones such as adrenaline, cortisol and norepinephrine, which can directly or indirectly alter neurotransmitter signaling in the central nervous system. For example, stress can reduce the levels of serotonin and dopamine in the brain. In some embodiments, stress sensitivity may precede the development of alcohol use disorder and contribute to susceptibility of the individual to developing the alcohol use disorder. For example, the alcohol use disorder may develop as a coping strategy (e.g., a self-medicating strategy) to treat stress. In some embodiments, stress sensitivity is a symptom caused by alcohol use disorder. For example, the alcohol use disorder may cause more stress, and a heightened sensitivity to stress. In some embodiments, stress sensitivity both precedes the development of alcohol use disorder, and is a symptom caused by the alcohol use disorder.

Alcohol use disorder includes behaviors that are locked into addictive patterns that are not awareness driven or life-goal-oriented. Denial of addiction is another barrier to treatment and recovery. Awareness training and mindfulness therapies have been associated with improved outcomes in substance abuse disorder. In embodiments, provided herein is a method for the treatment of AUD that reduces the preference for alcohol lowering the barrier to adjunct treatments for AUD. In certain embodiments, the compositions of this disclosure comprising a peptide product according to SEQ ID NO: 1, or a peptide product that is at least 80% identical to a peptide of SEQ ID NO: 1 and methods of treatment reduce the preference for alcohol and cravings thereof, thereby treating alcohol use disorder and/or alcohol dependence.

Mood and anxiety disorders are psychiatric disorders that are frequently comorbid with alcohol use disorder of this disclosure. Alcohol use disorder involve dysregulation of motivational circuits such as the dopamine reward circuitry, which are also implicated in mood and anxiety disorders. Alcohol use disorder also involves three major neurobiological circuits, the basal ganglia, the extended amygdala and the prefrontal cortex, which have also been implicated in mood and anxiety disorders. Mood disorders and anxiety disorders involve changes in a subject's mood. Exemplary mood disorders and anxiety disorders comprise depression, bipolar disorder, anxiety disorder, obsessive compulsive disorder and post-traumatic stress disorder. Signs of depression include, but are not limited to, a lack of interest in activities, changes in sleep pattern, changes in appetite, feelings of guilt, feelings of despair, lack of energy, trouble concentrating, stress, low mood, impaired motivation, cognitive impairment, diminished ability to think, anxiety, insomnia, anhedonia or negative affect, anorexia, fatigue and suicidal thoughts. Signs of anxiety include, but are not limited to, worry, distress, fear and panic attacks. Signs of bipolar disorder include, but are not limited to, drastic changes in mood, behavior and energy, sometimes termed episodes. Episodes can be manic, depressive or mixed. Bipolar disorder is also characterized mood swings. In some embodiments, alcohol use disorder can be thought of as involving three stages in a recurring cycle: (1) binge/intoxication, (2) withdrawal/negative affect, and (3) preoccupation/anticipation (craving). This cycle can worsen over time, and involves neuroplastic changes in the brain reward, stress, and executive function systems. Alcohol use disorder can also be thought of as chronically relapsing disorder, wherein shifts from impulsivity to compulsivity drive alcohol consumption. Thus, alcohol use disorder comprises symptoms also seen in obsessive-compulsive disorders. Obsessive-compulsive disorder is an anxiety disorder involving obsessions and compulsions. In embodiments provided herein, treatment of AUD with a composition of this disclosure reduces alcohol consumption and also treats co-morbid mood and anxiety disorders in the human subject.

In the treatment of psychiatric disorders associated with alcohol use disorder, such as depression, bipolar disorder, schizophrenia, anxiety disorders, post-traumatic stress disorder and autism spectrum disorder, medication is essential, and an antidepressant (e.g., a tricyclic antidepressant, a selective serotonin reuptake inhibitor, and a serotonin and norepinephrine reuptake inhibitor), an antipsychotic (e.g., a phenothiazine-based compound, a butyrophenone-based compound, a benzamide-based compound, an iminodibenzyl compound, a thiepin-based compound, an indole-based compound, and a serotonin/dopamine receptor antagonist), and an anti-anxiety drug are administered.

There are currently three main pharmacotherapeutic strategies used to treat substance use disorders: gradually reducing regimens, substitution therapies and medication assisted treatments. A subject with alcohol use disorder can be treated using a substitution therapy. In this case, the subject is given an agent, e.g. an agonist, which has a similar effect on the neural circuitry of the subject as the substance to which the individual is addicted. In some cases, the amount of this agent is then gradually reduced in a way that helps manage or reduce the severity of the withdrawal symptoms as use of the agent is discontinued. Exemplary agents used for this sort of therapy include, but are not limited to benzodiazepines for alcohol withdrawal, which have a similar effect on GABA.

Medication assisted treatments can also be used to treat symptoms, for example psychological symptoms, that contribute to relapse. Medication assisted treatments can target neurotransmitter systems that may be involved in heightened reactivity to substance related cues, stress sensitivity, aberrant reward processing or substance craving. These neurotransmitter systems include the glutamatergic, monoaminergic and opioid systems. Topiramate is a partial glutamate antagonist that reduces craving in alcohol dependence. Gabapentin increases the concentration of GABA in the brain and can be used to treat symptoms in alcohol users. Memantine, a low-affinity NMDAR antagonist, can reduce cue reactivity. Naltrexone, an opioid receptor antagonist, reduces cravings in a subject with alcohol dependence.

Medication assisted treatments, in addition to, or instead of, their effect on neurotransmission, can also act by changing the conditioned responses of a subject to alcohol, and affect reward processing. Disulfiram inhibits alcohol metabolism, leading to a buildup of toxic aldehydes with alcohol consumption. Disulfiram prevents the subject from receiving a pleasurable outcome from alcohol use, thus changing the reward salience of alcohol use.

Pharmacotherapies of the disclosure may be combined with behavioral therapies.

Exemplary, but non-limiting behavioral therapies comprise contingency management systems, counseling, mindfulness based therapies, cognitive behavioral therapy, digitally administered behavioral therapy or virtual reality based behavioral therapy. Counseling may be in person, or digitally administered (for example, web or telephone based counseling). Counseling may be one on one, e.g. with a therapist, with a group, or both. Contingency management is a type of behavioral therapy that uses operant conditioning. Contingency management provides rewards for desired behaviors (e.g., no substance use), and may take disciplinary measures in response to undesirable behaviors (e.g., substance use). Cognitive behavior therapy, originally developed to treat depression, has been expanded to treat symptoms of additional psychiatric and substance use disorders. In cognitive behavioral therapy, the subject works with one or more teachers or therapists to learn how to be aware of their thoughts and actions, and to identify and change mental distortions that contribute to psychiatric or substance use disorders. Mindfulness based therapy is a type of cognitive therapy that incorporates mindfulness practices such as meditation and breathing exercises. In some embodiments, mindfulness based therapy is a group therapy. In some embodiments, the behavioral therapy may be a virtual reality based therapy. Virtual reality based therapy have been used in treating symptoms such as paranoia and anxiety in subjects with psychiatric disorders. Virtual reality based therapies allow subjects to be exposed to, and respond to, stimuli in a controlled, virtual environment.

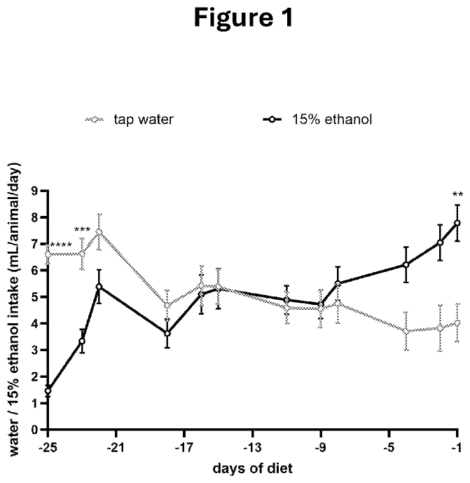

In certain embodiments, administering a therapeutically effective amount of a composition comprising a peptide product according to SEQ ID NO: 1, or a peptide product that is at least 80% identical to a peptide of SEQ ID NO: 1 of the disclosure can increase adherence to a treatment regimen. For example, over a 4-week induction period rodents slowly show a preference for ethanol when in an environment with free access to tap water or ethanol. See FIG. 1. However, administration of SEQ ID NO: 1 to the rodents showed that SEQ ID NO: 1 reduced the preference for ethanol compared to tap water within 2 days. See FIG. 6. This shows that administering a peptide product according to SEQ ID NO: 1, or a peptide product that is at least 80% identical to a peptide of SEQ ID NO: 1 can reduce the preference or liking of a subject for alcohol with an alcohol use disorder. This effect of compositions comprising a peptide product according to SEQ ID NO: 1, or a peptide product that is at least 80% identical to a peptide of SEQ ID NO: 1 of the disclosure, in addition to the ability of compositions comprising a peptide product according to SEQ ID NO: 1, or a peptide product that is at least 80% identical to a peptide of SEQ ID NO: 1 to reduce withdrawal symptoms as described herein, can increase the adherence to treatment regimens for alcohol use disorder, or decrease the addiction potential of alcohol. Accordingly, the compositions comprising a peptide product according to SEQ ID NO: 1, or a peptide product that is at least 80% identical to a peptide of SEQ ID NO: 1 and methods of the disclosure can increase adherence to a medically (medication) assisted treatment regimens for alcohol use disorder, such as medically assisted treatment for alcohol use disorder. The compositions comprising a peptide product according to SEQ ID NO: 1, or a peptide product that is at least 80% identical to a peptide of SEQ ID NO: 1 and methods of the disclosure can also increase the ability of those subjects who have stopped consuming alcohol to remain abstinent.

In certain embodiments provided herein are methods for prevention of relapse to alcohol dependence in a human being in need thereof comprising administering to the human being a once weekly therapeutic effective amount of a pharmaceutical dosage formulation comprising a peptide product according to SEQ ID NO: 1, or peptide product that is at least 80% identical to a peptide of SEQ ID NO: 1.

The disclosure further provides uses of the dual agonist peptide products described herein to prevent and/or treat conditions associated with GLP1R and/or GCGR (“ADU co-morbidity”), such as but not limited to insulin resistance, diabetes, obesity, metabolic syndrome and cardiovascular diseases, alcoholic fatty liver disease and PCOS. In some embodiments, the dual agonist peptide products can be used to treat hyperglycemia, insulin resistance, hyperinsulinemia, prediabetes, diabetes (including types 1 and 2, gestational and juvenile diabetes), diabetic complications, diabetic neuropathy, diabetic nephropathy, diabetic retinopathy, hyperlipidemia, hypercholesterolemia, hypertriglyceridemia, elevated blood levels of free fatty acids, obesity, metabolic syndrome, syndrome X, cardiovascular diseases (including coronary artery disease), atherosclerosis, acute cardiovascular syndrome, ischemia (including myocardial ischemia and cerebral ischemia/stroke), ischemia-reperfusion injury (including myocardial and cerebral IRI), infarction (including myocardial and cerebral infarction), angina, heart failure (e.g., congestive heart failure), peripheral vascular disease, thrombosis (e.g., deep vein thrombosis), embolism (e.g., pulmonary embolism), systemic inflammation (e.g., one characterized by elevated C-reactive protein blood level), and hypertension. The dual agonist peptide products can achieve their therapeutic effects through various mechanisms, including decrease in alcohol preference, stimulation of blood glucose-dependent insulin secretion, increase in insulin sensitivity, stimulation of fat burning and reduction of body weight.

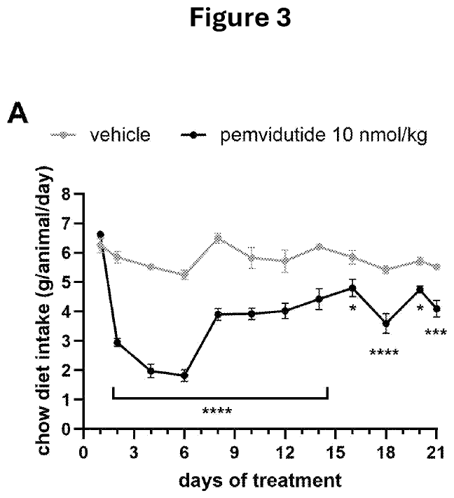

In certain embodiments, administering a therapeutically effective amount of a composition comprising a peptide product according to SEQ ID NO: 1, or a peptide product that is at least 80% identical to a peptide of SEQ ID NO: 1 of the disclosure can provide treatment to one or more AUD co-morbidities such as metabolic syndrome and/or disorders. For example, administration of SEQ ID NO: 1 to rodents showed that SEQ ID NO: 1 reduced mean body weight over a 3 week treatment period following a 4-week induction period to induce AUD in the rodents. See FIG. 2. That reduction in body weight corresponded with a reduced intake of the chow diet. See FIG. 3. This shows that administering compositions comprising a peptide product according to SEQ ID NO: 1, or a peptide product that is at least 80% identical to a peptide of SEQ ID NO: 1, in addition to reducing the preference or liking for alcohol, can also treat obesity or a subject that is overweight, in those subjects with AUD.

The peptide products described herein can be used to treat other conditions associated with AUD. Other conditions associated with AUD include without limitation arthritis (e.g., osteoarthritis), low back pain, breathing disorders (e.g., asthma, obesity hypoventilation syndrome [Pickwickian syndrome] and obstructive sleep apnea), dermatological disorders (e.g., diabetic ulcers, acanthosis nigricans, cellulitis, hirsutism, intertrigo and lymphedema), gastroenterological disorders (e.g., cholelithiasis [gallstone], gastroesophageal reflux disease [GERD] and gastroparesis), gout, hypercortisolism (e.g., Cushing's syndrome), kidney disorders (e.g., chronic kidney disease), liver disorders (e.g., alcoholic fatty liver disease [AFLD], metabolic dysfunction-associated steatotic liver disease [MetALD], alcohol-related liver disease [ALD], alcoholic steatohepatitis [ASH], alcohol-associated acute hepatitis [AH] or alcohol-associated cirrhosis [AC]), neurological disorders (e.g., carpal tunnel syndrome, dementias [e.g., Alzheimer's disease and vascular dementia], meralgia paresthetica, migraines and multiple sclerosis), urological disorders (e.g., erectile dysfunction, hypogonadism and urinary incontinence), polycystic ovary syndrome, infertility, menstrual disorders, mood disorders (e.g., depression), and cancers (e.g., cancers of the endometrium, esophagus, colorectum, gallbladder, kidney, liver [e.g., hepatocellular carcinoma], pancreas and skin [e.g., melanoma], breast and leukemia). In certain embodiments, a dual agonist peptide product described herein is used to treat polycystic ovary syndrome (PCOS). In other embodiments, a peptide product is used to treat chronic kidney disease (CKD), also known as chronic kidney/renal failure (CKF/CRF). The most common causes of CKD are diabetes and long-term, uncontrolled hypertension. In further embodiments, a dual agonist peptide product described herein is used to treat fatty liver disease (FLD). FLD, also known as hepatic steatosis, is characterized by excessive fat accumulation in the liver. FLD includes alcoholic fatty liver disease (AFLD). Chronic alcoholism causes fatty liver due to production of toxic metabolites such as aldehydes during metabolism of alcohol in the liver. Three stages of AFLD include: steatotic (fatty) liver; acute hepatitis and cirrhosis. Steatotic liver is the build-up of fat inside the liver cells. It leads to an enlarged liver. Steatotic liver is the most common alcohol-induced liver problem. Alcohol-associated hepatitis is an acute inflammation of the liver. There is death of liver cells, often followed by permanent scarring. Alcohol-associated cirrhosis is the destruction of normal liver tissue. It leaves scar tissue in place of the working liver tissue. In this situation, the liver may stop working correctly. Steatotic (fatty) liver can happen in anyone who consumes a lot of alcohol. Alcohol-associated hepatitis and cirrhosis are linked to long-term unhealthy alcohol use.

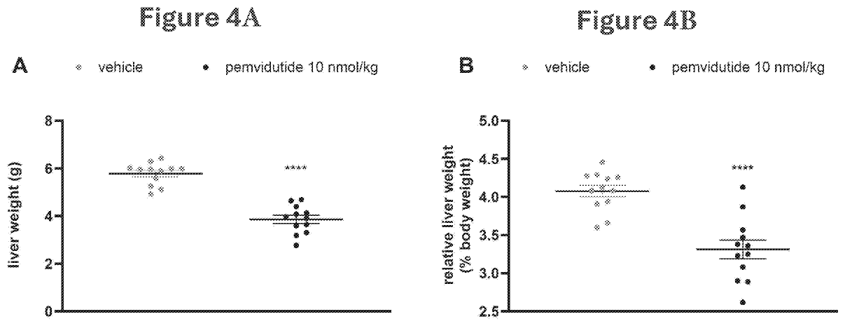

In certain embodiments, administering a therapeutically effective amount of a composition comprising a peptide product according to SEQ ID NO: 1, or a peptide product that is at least 80% identical to a peptide of SEQ ID NO: 1 of the disclosure can provide treatment to one or more conditions associated with AUD. For example, administration of SEQ ID NO: 1 to rodents showed that SEQ ID NO: 1 reduced liver weight (g) and relative liver weight (% body weight) over a 3 week treatment period following a 4-week induction period to induce AUD in the rodents. See FIG. 4. This shows that administering compositions comprising a peptide product according to SEQ ID NO: 1, or a peptide product that is at least 80% identical to a peptide of SEQ ID NO: 1, in addition to reducing the preference or liking for a alcohol, can also treat fatty liver disease, in those subjects with AUD.

AFLD represents an early hepatic injury with the following criteria: elevation in liver enzymes (aspartate aminotransferase [AST] or alanine aminotransferase [ALT]>35 U/L in men, AST or ALT>25 U/L in women), serum bilirubin <3 mg/dl, and the absence of other causes of liver disease. AFLD can be exacerbated by comorbidities such as diabetes, obesity and metabolic syndrome. Persistent alcohol use may lead to alcoholic steatohepatitis (ASH) characterized by steatosis accompanied by inflammation, hepatocyte ballooning, cell injury, and fibrosis. Fibrosis is the strongest predictor of mortality because it can lead to cirrhosis and heightened risk of hepatocellular carcinoma (HCC). HCC is the most common type of primary liver cancer in adults and occurs in the setting of chronic liver inflammation. AFLD affects up to 2.5-4.3% of the US population. Therefore, the peptide products described herein, can be used in the treatment of alcoholic fatty liver disease (AFLD) or alcoholic steatohepatitis (ASH) to mitigate liver steatosis, inflammation, fibrosis, and associated complications.

In some embodiments, the present dual agonist peptide(s) can be used in the treatment of AUD with reduction of one or more adverse events (i.e., an unexpected event that negatively impacts patient and/or animal welfare). Exemplary, non-limiting adverse events can include nausea, vomiting, diarrhea, abdominal pain and/or constipation. Adverse events may also include any known to those of ordinary skill in the art, such as those listed in industry resources and/or otherwise known to those of ordinary skill in the art (see, e.g., Medical Dictionary for Regulatory Activities (MedDRA) (Pharm., Med. Transl. Med. 2018) and/or Clark, M. J. Biomed. Inf., 54, April 2015, pp. 167-173). Such adverse events can be determined in humans using standard techniques as are typically used in clinical trials (e.g., doctor visit, surveys/questionnaires). As compared to the frequency and/or severity of such an adverse event that occurs upon administration of an agonist with unbalanced affinity for GLP-1R and GCGR (e.g., semaglutide) to a subject, the dual agonist peptides of this disclosure (e.g., any of SEQ ID NO. 1, or derivatives thereof) can decrease such frequency and/or severity thereof by, e.g., 5%, 10%, 20%, 40%, 50%, 60%, 70%, 80%, 90% of higher (up to 100%). In some embodiments, the dual agonist peptides of this disclosure (e.g., pemvidutide) do not cause any adverse events.

A present dual agonist peptide product can be administered by any suitable route for treatment of a condition or disorder disclosed herein. Potential routes of administration of a peptide product include without limitation oral, parenteral (including intradermal, subcutaneous, intramuscular, intravascular, intravenous, intra-arterial, intraperitoneal, intracavitary and topical), and topical (including transdermal, transmucosal, intranasal (e.g., by nasal spray or drop), ocular (e.g., by eye drop), pulmonary (e.g., by oral or nasal inhalation), buccal, sublingual, rectal (e.g., by suppository), and vaginal (e.g., by suppository)). In some embodiments, a peptide product is administered parenterally, such as subcutaneously, intravenously or intramuscularly. In other embodiments, a peptide product is administered by oral inhalation or nasal inhalation or insufflation. The therapeutically effective amount and the frequency of administration of, and the length of treatment with, a peptide product to treat a condition disclosed herein may depend on various factors, including the nature and severity of the condition, the potency of the compound, the route of administration, the age, body weight, general health, gender and diet of the subject, and the response of the subject to the treatment, and can be determined by the treating physician. In some embodiments, a peptide product is administered parenterally (e.g., subcutaneously (sc), intravenously (iv) or intramuscularly (im)) in a dose from about 0.01 mg to about 0.1, 1, 5 or 10 mg, or about 0.1-1 mg or 1-10 mg, over a period of about one week for treatment of a condition disclosed herein (e.g., one associated with AUD, such as AFLD or obesity). In further embodiments, a peptide product is administered parenterally (e.g., sc, iv or im) in a dose of about 0.1-0.5 mg, 0.5-1 mg, 1-5 mg or 5-10 mg over a period of about one week. In certain embodiments, a peptide product is administered parenterally (e.g., subcutaneously (SC), intravenous (IV) or intramuscular (IM)) in a dose of about 0.1-1 mg, or about 0.1-0.5 mg or 0.5-1 mg, over a period of about one week. One of skill in the art understands that an effective dose in a mouse, or other pre-clinical animal model, may be scaled for a human. In that way, through allometric scaling (also referred to as biological scaling) a dose in a larger animal may be extrapolated from a dose in a mouse to obtain an equivalent dose based on body weight or body surface area of the animal.

The pemvidutide formulation can be taken substantially with food (e.g., with a meal or within about 1 hour or 30 minutes before or after a meal) or substantially without food (e.g., at least about 1 or 2 hours before or after a meal). The length of treatment of a medical condition with a dual agonist peptide product can be based on, e.g., the nature and severity of the condition and the response of the subject to the treatment and can be determined by the treating physician. In some embodiments, the pemvidutide formulation is administered chronically to treat a condition disclosed herein, such as at least about 2 months, 3 months, 6 months, 1 year, 1.5 years, 2 years, 3 years, 5 years, 10 years or longer. The pemvidutide formulation can also be taken pro re nata (as needed) until clinical manifestations of the condition disappear or clinical targets are achieved, such as blood glucose level, blood pressure, blood levels of lipids, body weight or body mass index, waist-to-hip ratio or percent body fat, or any combination thereof. If clinical manifestations of the condition re-appear or the clinical targets are not maintained, administration of the dual agonist peptide product can resume. The disclosure provides a method of treating a medical condition described herein, comprising administering to a subject in need of treatment a therapeutically effective amount of the pemvidutide formulation described herein or a pharmaceutically acceptable salt thereof, or a pharmaceutical composition comprising the same. The disclosure further provides pemvidutide described herein or a pharmaceutically acceptable salt thereof, or a composition comprising the same, for use as a medicament. In addition, the disclosure provides for the use of pemvidutide described herein or a pharmaceutically acceptable salt thereof in the preparation of a medicament. The medicament containing pemvidutide can be used to treat any medical condition described herein. The pemvidutide can optionally be used in combination with one or more additional therapeutic agents.

Pemvidutide (e.g., as a formulation) can be administered as the sole active agent, or optionally be used in combination with one or more other dual agonist peptide products, and/or additional therapeutic agents (as described above) in the treatment of AUD, and disorders associated thereof, such as insulin resistance, diabetes, obesity, metabolic syndrome, mood disorders or a cardiovascular disease, or any condition associated therewith. In some embodiments, the one or more additional therapeutic agents are selected from antidiabetic agents, anti-obesity agents (including lipid-lowering agents and pro-satiety agents), anti-atherosclerotic agents, anti-inflammatory agents, antioxidants, antifibrotic agents, anti-hypertensive agents, and combinations thereof. Antidiabetic agents include without limitation: AMP-activated protein kinase (AMPK) agonists, including biguanides (e g., buformin and metformin); peroxisome proliferator-activated receptor gamma (PPAR-γ) agonists, including thiazolidinediones (e.g., balaglitazone, ciglitazone, darglitazone, englitazone, lobeglitazone, netoglitazone, pioglitazone, rivoglitazone, rosiglitazone and troglitazone), MSDC-0602K and saroglitazar (dual PPAR-α/γ agonist); glucagon-like peptide-1 (GLP-1) receptor agonists, including exendin-4, albiglutide, dulaglutide, exenatide, liraglutide, lixisenatide, semaglutide, taspoglutide, CNT0736, CNT03649, HM11260C (LAPS-Exendin), NN9926 (OG9S7GT), TT401 and ZYOG1; dipeptidyl peptidase 4 (DPP-4) inhibitors, including alogliptin, anagliptin, dutogliptin, evogliptin, gemigliptin, gosogliptin, linagliptin, omarigliptin, saxagliptin, septagliptin, sitagliptin, teneligliptin, trelagliptin and vildagliptin; sodium-glucose transport protein 2 (SGLT2) inhibitors, including canagliflozin (also inhibits SGLT1), dapagliflozin, empagliflozin, ertugliflozin, ipragliflozin, luseogliflozin, remogliflozin etabonate, sotagliflozin (also inhibits SGLT1) and tofogliflozin; blockers of ATP-dependent K⁺ (KA_{TP}) channels on pancreatic beta cells, including rneglitinides (e.g., mitiglinide, nateglinide and repagiinide) and sulfonylureas {including first generation (e.g., acetohexamide, carbutamide, chlorpropamide, giycyclamide [tolhexamide], metahexamide, tolazamide and tolbutamide) and second generation (e.g., glibenclamide, glyburide, glibornuride, gliclazide, glimepiride, glipizide, gliquidone, glisoxepide and glyclopyramide); insulin and analogs thereof, including fast-acting insulin (e.g., insulin aspari insulin glulisine and insulin lispro), intermediate-acting insulin (e.g., NPH insulin), and long-acting insulin (e.g., insulin degludec, insulin detemir and insulin glargine); and/or, analogs, derivatives and salts thereof. In certain embodiments, the antidiabetic agent is or includes a biguanide (e.g., metformin), a thiazolidinedione (e.g., pioglitazone or rosiglitazone) or a SGLT2 inhibitor (e.g., empagliflozin or tofogliflozin), or any combination thereof. Anti-obesity agents include, but are not limited to: appetite suppressants (anorectics), including amphetamine, dexamphetamine, amfepramone, clobenzorex, mazindol, phentermine (with or without topiramate) and lorcaserin; pro-satiety agents, including ciliary neurotrophic factor (e.g., axokine) and longer-acting analogs of amylin, calcitonin, cholecystokinin (CCK), GLP-1, leptin, oxyntomodulin, pancreatic polypeptide (PP), peptide YY (PYY) and neuropeptide Y (NPY); lipase inhibitors, including caulerpenyne, cetilistat, ebelactone A and B, esterastin, lipstatin, orlistat, percyquinin, panclicin A-E, valilactone and vibralactone; antihyperlipidemic agents; and analogs, derivatives and salts thereof. Antihyperlipidemic agents include without limitation: HMG-COA reductase inhibitors, including statins {e.g., atorvastatin, cerivastatin, fluvastatin, mevastatin, monacolins (e.g., monacolin K (lovastatin), pitavastatin, pravastatin, rosuvastatin and simvastatin} and flavanones (e.g., naringenin); squalene synthase inhibitors, including lapaquistat, zaragozic acid and RPR-107393; acetyl-CoA carboxylase (ACC) inhibitors, including anthocyanins, avenaciolides, chloroacetylated biotin, cyclodim, diclofop, haloxyfop, soraphens (e.g., soraphen Ala), 5-(tetradecyloxy)-2-furancarboxylic acid (TOFA), CP-640186, GS-0976, NDI-010976; 7-(4-propyloxy-phenylethynyl)-3,3-dimethyl-3,4dihydro-2H-benzo[b][1,4]dioxepine; N-ethyl-N′-(3-{[4-(3,3-dimethyl-1-oxo-2-oxa-7-azaspiro[4.5]dec-7-yl) piperidin-1-yl]-carbonyl}-1-benzothien-2-yl)urea; 5-(3-acetamidobut-1-ynyl)-2-(4-propyloxyphenoxy) thiazole; and 1-(3-{[4-(3,3-dimethyl-1-oxo-2-oxa-7-azaspiro[4.5]dec-7-yl) piperidin-1-yl]-carbonyl}-5-(pyridin-2-yl)-2-thienyl)-3-ethylurea; PPAR-α agonists, including fibrates (e.g., bezafibrate, ciprofibrate, clinofibrate, clofibric acid, clofibrate, aluminum clofibrate [alfibrate], clofibride, etofibrate, fenofibric acid, fenofibrate, gemfibrozil, ronifibrate and simfibrate), isoflavones (e.g., daidzein and genistein), and perfluoroalkanoic acids (e.g., perfluorooctanoic acid and perfluorononanoic acid); PPAR-δ agonists, including elafibranor (dual PPAR-α/γ agonist), GFT505 (dual PPAR-α/γ agonist), GW0742, GW501516 (dual PPAR-β/δ agonist), sodelglitazar (GW677954), MBX-8025, and isoflavones (e.g., daidzein and genistein); PPAR-γ agonists, including thiazolidinediones {supra), saroglitazar (dual PPAR-α/γ agonist), 4-oxo-2-thioxothiazolines (e.g., rhodanine), berberine, honokiol, perfluorononanoic acid, cyclopentenone prostaglandins (e.g., cyclopentenone 15-deoxy-A-prostaglandin J₂ [15d-PGJ₂]), and isoflavones (e.g., daidzein and genistein); liver X receptor (LXR) agonists, including endogenous ligands (e.g., oxysterols such as 22(i?)-hydroxycholesterol, 24(A)-hydroxy cholesterol, 27-hydroxycholesterol and cholestenoic acid) and synthetic agonists (e.g., acetyl-podocarpic dimer, hypocholamide, A (X-di methyl-3 b-hydroxy-cholenamide [DMHCA], GW3965 and T0901317); retinoid X receptor (RXR) agonists, including endogenous ligands (e.g., 9-cis-retinoic acid) and synthetic agonists (e.g., bexarotene, AGN 191659, AGN 191701, AGN 192849, BMS649, LG100268, LG100754 and LGD346); inhibitors of acyl-CoA cholesterol acyltransferase (ACAT, aka sterol G-acyl transferase [SOAT], including ACAT1 [SOAT1] and ACAT2 [SOAT2]), including avasimibe, pactimibe, pellitorine, terpendole C and flavanones (e.g., naringenin); inhibitors of stearoyl-CoA desaturase-1 (SCD-1, aka stearoyl-CoA delta-9 desaturase) activity or expression, including aramchol, CAY-10566, CVT-11127, SAR-224, SAR-707, XEN-103; 3-(2-hydroxyethoxy)-4-methoxy-N-[5-(3-trifluoromethylbenzyl) thiazol-2-yl]benzamide and 4-ethylamino-3-(2-hydroxyethoxy)-N-[5-(3-trifluoromethylbenzyl) thiazol-2-yl]benzamide; 1′-{6-[5-(pyridin-3-ylmethyl)-1,3,4-oxadiazol-2-yl]pyridazin-3-yl}-5-(trifluoromethyl)-3,4-dihydrospiro[chromene-2,4′-piperidine]; 5-fluoro-1′-{6-[5-(pyridin-3-ylmethyl)-1,3,4-oxadiazol-2-yl]pyridazin-3-yl}-3,4-dihydrospiro[chromene-2,4′-piperidine]; 6-[5-(cyclopropylmethyl)-4,5-dihydro-1′H,3H-spiro[1,5-benzoxazepine-2,4′-piperidin]-1′-yl]-N-(2-hydroxy-2-pyridin-3-ylethyl)pyridazine-3-carboxamide; 6-[4-(2-methylbenzoyl) piperidin-1-yl]pyridazine-3-carboxylic acid (2-hydroxy-2-pyridin-3-ylethyl)amide; 4-(2-chlorophenoxy)-N-[3-(methyl carbamoyl)phenyl]piperidine-1-carboxamide; the cis-9, trans-11 isomer and the trans-10,cis-12 isomer of conjugated linoleic acid, substituted heteroaromatic compounds disclosed in WO 2009/129625 A1, anti-sense polynucleotides and peptide-nucleic acids (PNAs) that target mRNA for SCD-1, and SCD-1-targeting siRNAs; cholesterylester transfer protein (CETP) inhibitors, including anacetrapib, dalcetrapib, evacetrapib, torcetrapib and AMG 899 (TA-8995); inhibitors of microsomal triglyceride transfer protein (MTTP) activity or expression, including implitapide, lomitapide, dirlotapide, mitratapide, CP-346086, JTT-130, SLx-4090, anti-sense polynucleotides and PNAs that target mRNA for MTTP, MTTP-targeting microRNAs (e.g., miRNA-30c), and MTTP-targeting siRNAs; GLP-1 receptor agonists; fibroblast growth factor 21 (FGF21) and analogs and derivatives thereof, including BMS-986036 (pegylated FGF21); inhibitors of pro-protein convertase subtilisin/kexin type 9 (PCSK9) activity or expression, including berberine (reduces PC8K9 level), annexin A2 (inhibits PCSK9 activity), anti-PCSK9 antibodies (e.g., alirocumab, bococizumab, evolocumab, LGT-209, LY3015014 and RG7652), peptides that mimic the epidermal growth factor-A (EGF-A) domain of the LDL receptor which binds to PCSK9, PCSK9-binding adnectins (e.g., BMS-962476), anti-sense polynucleotides and PNAs that target mRNA for PCSK9, and PCSK9-targeting siRNAs (e.g, inclisiran [ALN-PCS] and ALN-PCS02); apolipoprotein mimetic peptides, including apoA-I mimetics (e.g., 2F, 3F, 3F-1, 3F-2, 3F-14, 4F, 4F-P-4F, 4F-IHS-4F, 4F2, 5F, 6F, 7F, 18F, 5A, 5A-C1, 5A-CH1, 5A-CH2, 5A-H1, 18 A, 37 pA [18A-P-18A], ELK, ELK-1A, ELK-1F, ELK-1K1A1E, ELK-1L1K, ELK-1W, ELK-2A, ELK-2A2K2E, ELK-2E2K, ELK-2F, ELK-3 E3EK, ELK-3E3K3A, ELK-3E3LK, ELK-PA, ELK-P2A, ELKA, ELKA-CH2, ATI-5261, CS-6253, ETC-642, FAMP, FREL and KRES and apoE mimetics (e.g., Ac-hE18A-NH₂, AEM-28, Ac-[R]hEl 8 A-NH₂, AEM-28-14, EpK, hEp, mR18L, COG-112, COG-133 and COG-1410); omega-3 fatty acids, including docosahexaenoic acid (DHA), docosapentaenoic acid (DPA), eicosapentaenoic acid (EPA), α-linolenic acid (ALA), fish oils (which contain, e.g., DHA and EPA), and esters (e.g., glyceryl and ethyl esters) thereof; and analogs, derivatives and salts thereof. In certain embodiments, the anti-obesity agent is or includes a lipase inhibitor (e.g., orlistat) or/and an antihyperlipidemic agent (e.g., a statin such as atorvastatin, or/and a fibrate such as fenofibrate). Antihypertensive agents include without limitation: antagonists of the renin-angiotensin-aldosterone system (RAAS), including renin inhibitors (e.g., aliskiren), angiotensin-converting enzyme (ACE) inhibitors (e.g., benazepril, captopril, enalapril, fosinopril, lisinopril, moexipril, perindopril, quinapril, ramipril and trandolapril), angiotensin II receptor type 1 (ATII1) antagonists (e.g., azilsartan, candesartan, eprosartan, fimasartan, irbesartan, losartan, olmesartan medoxomil, olmesartan, telmisartan and valsartan), and aldosterone receptor antagonists (e.g., eplerenone and spironolactone); diuretics, including loop diuretics (e.g., bumetanide, ethacrynic acid, furosemide and torsemide), thiazide diuretics (e.g., bendroflumethiazide, chlorothiazide, hydrochlorothiazide, epitizide, methyclothiazide and polythiazide), thiazide-like diuretics (e.g., chlorthalidone, indapamide and metolazone), cicletanine (an early distal tubular diuretic), potassium-sparing diuretics (e.g., amiloride, eplerenone, spironolactone and triamterene), and theobromine; calcium channel blockers, including dihydropyridines (e.g., amlodipine, levamlodipine, cilnidipine, clevidipine, felodipine, isradipine, lercanidipine, nicardipine, nifedipine, nimodipine, nisoldipine and nitrendipine) and non-dihydropyridines (e.g., diltiazem and verapamil); α2-adrenoreceptor agonists, including clonidine, guanabenz, guanfacine, methyldopa and moxonidine; α1-adrenoreceptor antagonists (alpha blockers), including doxazosin, indoramin, nicergoline, phenoxybenzamine, phentolamine, prazosin, terazosin and tolazoline; β-adrenoreceptor (β1 or/and β2) antagonists (beta blockers), including atenolol, betaxolol, bisoprolol, carteolol, carvedilol, labetalol, metoprolol, nadolol, nebivolol, oxprenolol, penbutolol, pindolol, propranolol and timolol; mixed alpha/beta blockers, including bucindolol, carvedilol and labetalol; endothelin receptor antagonists, including selective ETA receptor antagonists (e.g., ambrisentan, atrasentan, edonentan, sitaxentan, zibotentan and BQ-123) and dual ET_{A}/ET_{B} antagonists (e.g., bosentan, macitentan and tezosentan); other vasodilators, including hydralazine, minoxidil, theobromine, sodium nitroprusside, organic nitrates (e.g., isosorbide mononitrate, isosorbide dinitrate and nitroglycerin, which are converted to nitric oxide in the body), endothelial nitric oxide synthase (eNOS) stimulators (e.g., cicletanine), activators of soluble guanylate cyclase (e.g., cinaciguat and riociguat), phosphodiesterase type 5 (PDE5) inhibitors (e.g., avanafil, benzamidenafil, dasantafil, dynafil, lodenafil, mirodenafil, sildenafil, tadalafil, udenafil, vardenafil, dipyridamole, papaverine, propentofylline, zaprinast and T-1032), prostaglandin Ei (alprostadil) and analogs thereof (e.g., limaprost amd misoprostol), prostacyclin and analogs thereof (e.g., ataprost, beraprost [e.g., esuberaprost], 5,6,7-trinor-4,8-inter-w-phenylene-9-fluoro-PGl₂, carbacyclin, isocarbacyclin, clinprost, ciprostene, eptaloprost, cicaprost, iloprost, pimilprost, SM-10906 (des-methyl pimilprost), naxaprostene, taprostene, treprostinil, CS-570, OP-2507 and TY-11223), non prostanoid prostacyclin receptor agonists (e.g., 1-phthalazinol, ralinepag, selexipag, ACT-333679 [MRE-269, active metabolite of selexipag], and TRA-418), phospholipase C (PLC) inhibitors, and protein kinase C (PKC) inhibitors (e.g., BIM-1, BIM-2, BIM-3, BIM-8, chelerythrine, cicletanine, gossypol, miyabenol C, myricitrin, ruboxistaurin and verbascoside; minerals, including magnesium and magnesium sulfate; and analogs, derivatives and salts thereof. In certain embodiments, the antihypertensive agent is or includes a thiazide or thiazide like diuretic (e.g., hydrochlorothiazide or chlorthalidone), a calcium channel blocker (e.g., amlodipine or nifedipine), an ACE inhibitor (e.g., benazepril, captopril or perindopril) or an angiotensin II receptor antagonist (e.g., olmesartan medoxomil, olmesartan, telmisartan or valsartan), or any combination thereof.

In some embodiments, a peptide product described herein is used in combination with one or more additional therapeutic agents to treat fatty liver and/or liver fibrosis. In some embodiments, the one or more additional therapeutic agents are selected from antidiabetic agents, anti-obesity agents, anti-inflammatory agents, antifibrotic agents, antioxidants, anti hypertensive agents, and combinations thereof. Therapeutic agents that can be used to treat fatty liver and/or liver fibrosis include without limitation: PPAR agonists, including PPAR-δ agonists (e.g., MBX-8025, elafibranor [dual PPAR-α/δ agonist] and GW501516 [dual PPAR-β/δ agonist]) and PPAR-γ agonists (e.g., thiazolidinediones such as pioglitazone, and saroglitazar [dual PPAR-α/γ agonist])-PPAR-δ and -γ agonism increases insulin sensitivity, PPAR-α agonism reduces liver steatosis and PPAR-δ agonism inhibits activation of macrophages and Kupffer cells; farnesoid X receptor (FXR) agonists, such as obeticholic acid and nonsteroidal FXR agonists like GS-9674 reduce liver gluconeogenesis, lipogenesis, steatosis and fibrosis; fibroblast growth factor 19 (FGF19) and analogs and derivatives thereof, such as NGM-282-FGF19 analogs reduce liver gluconeogenesis and steatosis; fibroblast growth factor 21 (FGF21) and analogs and derivatives thereof, such as BMS-986036 (pegylated FGF21)-FGF21 analogs reduce liver steatosis, cell injury and fibrosis; HMG-COA reductase inhibitors, including statins (e.g., rosuvastatin)—statins reduce steatohepatitis and fibrosis; ACC inhibitors, such as NDI-010976 (liver-targeted) and GS-0976-ACC inhibitors reduce de novo lipogenesis and liver steatosis; SCD-1 inhibitors, such as aramchol-SCD-1 inhibitors reduce liver steatosis and increase insulin sensitivity; SGLT2 inhibitors, such as canagliflozin, ipragliflozin and luseogliflozin-SGLT2 inhibitors reduce body weight, liver ALT level and fibrosis; antagonists of CCR2 or/and CCR5, such as cenicriviroc-antagonists of CCR2 (binds to CCL2 [MCP1]) and CCR5 (binds to CCL5 [RANTES]) inhibit activation and migration of inflammatory cells (e.g., macrophages) to the liver and reduce liver fibrosis; apoptosis inhibitors, including apoptosis signal-regulating kinase 1 (ASK1) inhibitors (e.g., selonsertib) and caspase inhibitors (e.g., emricasan [pan-caspase inhibitor])—apoptosis inhibitors reduce liver steatosis and fibrosis; lysyl oxidase-like 2 (LOXL2) inhibitors, such as simtuzumab—LOXL2 is a key matrix enzyme in collagen formation and is highly expressed in the liver; galectin-3 inhibitors, such as GR-MD-02 and TD139—galectin-3 is critical for development of liver fibrosis; antioxidants, including vitamin E (e.g., α-tocopherol) and scavengers of reactive oxygen species (ROS) and free radicals (e.g., cysteamine, glutathione, melatonin and pentoxifylline [also anti-inflammatory via inhibition of TNF-α and phosphodiesterases])—vitamin E reduces liver steatosis, hepatocyte ballooning and lobular inflammation; and, analogs, derivatives and salts thereof. In some embodiments, a peptide product described herein is used in conjunction with a PPAR agonist (e.g., a PPAR-δ agonist such as elafibranor or/and a PPAR-γ agonist such as pioglitazone), a HMG-COA reductase inhibitor (e.g., a statin such as rosuvastatin), an FXR agonist (e.g., obeticholic acid) or an antioxidant (e.g., vitamin E), or any combination thereof, to treat fatty liver and/or liver fibrosis. In certain embodiments, the one or more additional therapeutic agents for treatment of fatty liver and/or liver fibrosis are or include vitamin E or/and pioglitazone. Other combinations may also be used as would be understood by those of ordinary skill in the art.

Pharmacokinetic (“PK”) parameters can be estimated using Phoenix® WinNonlin® version 8.1 or higher (Certara USA, Inc., Princeton, New Jersey). A non-compartmental approach consistent with the extravascular route of administration can be used for parameter estimation. The individual plasma concentration-time data can be used for pharmacokinetic calculations. In addition to parameter estimates for individual animals, descriptive statistics (e.g. mean, standard deviation, coefficient of variation, median, min, max) can be determined, as appropriate. Concentration values that are below the limit of quantitation can be treated as zero for determination of descriptive statistics and pharmacokinetic analysis. Embedded concentration values that are below the limit of quantitation can be excluded from pharmacokinetic analysis. All parameters can be generated from individual dual agonist peptide (or derivatives and/or metabolites thereof) concentrations in plasma from test article-treated groups on the day of dosing (Day 1). Parameters can be estimated using nominal dose levels, unless out of specification dose formulation analysis results are obtained, in which case actual dose levels can be used. Parameters can be estimated using nominal sampling times; if bioanalytical sample collection deviations are documented, actual sampling times can be used at the affected time points. Bioanalytical data can be used as received for the pharmacokinetic analysis and can be presented in tables and figures in the units provided. Pharmacokinetic parameters can be calculated and presented in the units provided by the analytical laboratory (the order of magnitude can be adjusted appropriately for presentation in the report, e.g. h*ng/ml converted to h*ug/mL). Descriptive statistics (e.g., mean, standard deviation, coefficient of variation, median, min, max) and pharmacokinetic parameters can be determined to three significant figures, as appropriate. Additional data handling items can be documented as needed. PK parameters to be determined, as data permit, can include but are not limited to the following: Cₘₐₓ: Maximum observed concentration; DN Cₘₐₓ: dose normalized maximum concentration, calculated as Cₘₐₓ/dose; Tₘₐₓ: time of maximum observed concentration; AUC₀₋ₜ: area under the curve from time 0 to the time of the last measurable concentration, calculated using the linear trapezoidal rule; AUC₀₋₉₆: area under the curve from time 0 to hour 96, calculated using the linear trapezoidal rule; DN AUC₀₋₉₆: dose normalized AUC₀₋₉₆, calculated as AUC₀₋₉₆/dose; AUC_{0-inf}: area under the curve from time 0 to infinity (Day 1 only), calculated as AUC_{0-inf}=AUC₀₋ₜ+Cₜ/λ_{z}, where Cₜ is the last observed quantifiable concentration and λ_{z} is the elimination rate constant; t_{1/2}: elimination half-life, calculated as ln(2)/λ_{z}. Additional parameters and comparisons (e.g., sex ratios, dose proportionality ratios, etc.) can also be determined, as would be understood by those of ordinary skill in the art.

In some embodiments, this disclosure provides pharmaceutical dosage formulation(s) comprising pemvidutide wherein the peptide product is modified with a hydrophobic surfactant; the dosage is configured to reduce alcohol consumption and/or maintained reduced alcohol consumption, with reduction of one or more adverse events wherein the human in need thereof has AUD and may also be overweight, obese, and/or suffer from type 2 diabetes, wherein the adverse events being selected from nausea, vomiting, diarrhea, abdominal pain and constipation, upon administration to a mammal. In certain embodiments, the formulation comprises 1.2, 1.8, or 2.4 mg of pemvidutide as a weekly therapeutic dose (the 2.4 mg dose being preferably preceded by titration up to 2.4 mg for four weeks (preferably 0.6 mg during week 1, 1.2 mg per week during week 2, 1.8 mg per week during week 3, and 1.8 mg per week during week 4), preferably weekly for 48 weeks. In certain embodiments, the formulation comprises 1.2, 1.8, or 2.4 mg of pemvidutide as a weekly therapeutic dose, the 2.4 mg dose being preferably preceded by titration up to 2.4 mg with 1.2 mg once weekly for 4 weeks, 1.8 mg once weekly for 4 weeks, and 2.4 mg once weekly for subsequent dosing.

“Reducing,” or “reduction of” adverse effects or events refers to a reduction in the degree, duration, and/or frequency of adverse effects experienced by a subject and incidence in a group of subjects following administration of an agonist with about balanced affinity to GLP1R and GCGR compared to an agonist with unbalanced affinity for GLP1R and GCGR. Such reduction encompasses the prevention of some adverse effects that a subject would otherwise experience in response to an agonist with unbalanced affinity to GLP1R and GCGR. Such reduction also encompasses the elimination of adverse effects previously experienced by a subject following administration of an agonist with unbalanced affinity to GLP1R and GCGR. In some embodiments, “reducing,” or “reduction of” adverse effects encompass a reduction of gastrointestinal side effects wherein the adverse events are reduced to zero or undetectable levels. In other embodiments, adverse effect is reduced to level equivalent to untreated subjects but not completely eliminated. Moreover, administration of analogs with unbalanced affinity toward GLP-1R or GCGR to a mammal may lead to the need for an excessively high dose to maximally activate the receptor with weaker sensitivity toward the ligand, thus leading to a potential for exceeding the biologically effective dose level for the other ligand and causing dose-related, undesired side effects.

In certain embodiments, this disclosure provides methods in the treatment of AUD that reduce body weight in a human being, wherein the method comprises administering pemvidutide once weekly in an amount from at least 1.2 mg up to 2.4 mg (preferably 1.2 mg, 1.8 mg, or 2.4 mg with titration up to 2.4 mg) to the human being in need thereof; and, wherein the human being is overweight, obese, and/or may also suffer from type 2 diabetes. In some preferred embodiments of such methods, the pemvidutide is administered once weekly in an amount of 2.4 mg (following titration to the 2.4 mg dose) and the body weight of the human being decreases by at least about 5%, at least 10% or at least 15%. In preferred embodiments, the patients exhibit substantial and clinically meaningful reductions in one or more of total cholesterol, low density lipoproteins, triglycerides, and/or blood pressure. In preferred embodiments, within a population of patients to whom the 2.4 mg weekly dose has been administered, at least about 30% or more of the patients lose about least about 20% or more body weight at week 48. In preferred embodiments, continued administration of the 2.4 weekly dose, or in some embodiments a lesser dose, continues to induce weight loss in a patient until a desired weight is achieved. In some embodiments, a maintenance dose (e.g., in preferred embodiments, a 1.2, 1.8 or 2.4 mg weekly dose) may be administered to the patient, either after the initial 48-week administration is completed, or after the desired weight is achieved. In preferred embodiments, the patient(s) experience no gastrointestinal (GI) or cardiac (e.g., arrythmia, increased heart rate) adverse events. In preferred embodiments, where a patient(s) experiences GI adverse events, such event is considered by the patient and/or an attending medical professional as mild to moderate in severity. In preferred embodiments, the patient also exhibits stable glucose homeostasis.

In some embodiments, this disclosure provides pharmaceutical dosage formulations configured for administering to the human the agonist peptide product (e.g., SEQ ID NO: 1) at less than about 0.15 mg/kg/dose, optionally from about 0.03 to 0.10 mg/kg/dose. In some embodiments, the pharmaceutical dosage formulation is configured to administer less than 0.15 mg/kg/dose of the agonist peptide product to the mammal. In some embodiments, the pharmaceutical dosage formulation configured to administer between 0.03-0.15 mg/kg/dose. In some embodiments, the pharmaceutical dosage formulation can be configured to administer between about 0.5 to about 10 mg per week; optionally about 1 to about 7 mg per week; or optionally about 1 to 5 mg per week. In some embodiments, the pharmaceutical dosage formulation is configured to be administered to the mammal once weekly for up to six weeks. In some embodiments, this disclosure provides pharmaceutical dosage formulations configured such that the time to reach a therapeutic dose is about four weeks or less. In some embodiments, the therapeutic dose exhibits a Cₘₐₓ of from about 20 to about 200 ng/ml; a Tₘₐₓ of from about 20 to about 80 hours; and/or, an AUC₀₋ₜ of from about 5,000 to 40,000 h*ng/mL.

In some embodiments, the methods disclosed herein do not comprise a treatment initiation phase. In other words, the first administered dose is therapeutic without the need to titrate to avoid adverse gastrointestinal side effects. For instance, in some embodiments, the method can comprise administering a first one or more doses (the treatment initiation phase) of a peptide of this disclosure, such as SEQ ID NO: 1, followed by subsequent second one or more and higher doses of the peptide, each of the first and second doses being administered for one or more weeks. In some embodiments, the first dose(s) and the second dose(s) can be followed by one or more third doses that can be higher than the second dose(s). The switch from the first dose, the second dose, and the third dose can be made on a weekly basis. For instance, if it appears the first dose has not induced lower blood glucose and/or weight loss after one or more weeks, the second higher dose can then be administered for one or more weeks followed by an analysis of the effects of the second dose(s). If the beneficial effects are observed (e.g., reduced preference for alcohol), the second dose can continue to be administered. If the beneficial effects are not observed, the third dose may be administered for one or more weeks, followed by a determination of beneficial effects. This cycle of dosing and analysis can be repeated as appropriate, provided adverse events are not observed with each dose. In some embodiments, the subsequent second one or more and higher doses of the peptide can be administered because reduced alcohol preference was not achieved after about four weeks of administration of the first one or more doses. In some embodiments, the first one or more doses can be administered without the intention to produce a therapeutic effect (e.g., reduced preference for alcohol). In some embodiments, however, the methods can be carried out without including the treatment initiation phase.

In some embodiments, the methods can be a first line indication for alcohol use disorder (AUD) in a human being, meaning that it is the first and sole active agent administered to the patient for the purpose of treating AUD in the human being. In some embodiments, the methods disclosed herein can include an adjunct treatment of known AUD therapeutics (e.g., Disulfiram (Antabuse®)). In some embodiments, the human being to whom the pharmaceutical dosage is administered has obesity. In some embodiments, the human being to whom the pharmaceutical dosage is administered has fatty liver disease.

In some embodiments, the pharmaceutical dosage is administered about weekly. In some embodiments, the pharmaceutical dosage is administered to the human being about weekly from about 2 weeks to about 8 weeks, or longer. In some embodiments, the pharmaceutical dosage is administered on about days 1, 8, 15, 22, 29, and 36. In some embodiments, the methods can include administering the pharmaceutical dosage to an adult human at from about 0.5 mg/dose, about 1.0 mg/dose, about 1.5 mg/dose, about 2.0 mg/dose, about 2.5 mg/dose, about 3.0 mg/dose, about 3.5 mg/dose, about 4.0 mg/dose, about 4.5 mg/dose, about 5.0 mg/dose, or about 5.5 mg/dose. In some embodiments, the pharmaceutical dosage can be administered about once per week or once every two weeks, optionally for at least one month; optionally wherein each dose comprises about the same amount of agonist peptide product. In some embodiments, the pharmaceutical dosage can be administered subcutaneously. In some embodiments, one or more of the doses can be administered via a first route (e.g., subcutaneously) and subsequently administered by a different route (e.g., orally). In some embodiments, the time to reach a therapeutic dose is about four weeks or less. In some embodiments, administration of the pharmaceutical dosage formulation exhibits a Cₘₐₓ of from about 400 to about 1300 ng/ml; a Tₘₐₓ of from about 10 to about 36 hours; and/or, an AUC₀₋₄₈ of from about 15,000 to 45,000 h*ng/mL. In certain embodiments, the weight loss in the human being is at least 5%, at least 10%; or from about 1% to about 20%; or from about 5% to about 10% (w/w). In some embodiments, administration of a pharmaceutical dosage formulation comprising about 1.8 mg of a peptide product according to SEQ ID NO: 1, or a peptide product that is at least 80% identical to a peptide of SEQ ID NO: 1 is administered (preferably subcutaneously) once per week for at least six weeks results in greater than about 3-5% whole-body weight loss, preferably greater than about 5% over a population to whom the formulation was administered (“mean weight loss”), and/or a net change from placebo of about 6%, preferably about 6.3%. In some embodiments, administration of a pharmaceutical dosage formulation comprising about 1.2 mg of a peptide product according to SEQ ID NO: 1, or a peptide product that is at least 80% identical to a peptide of SEQ ID NO: 1 is administered (preferably subcutaneously) once per week for at least six weeks results in about 2% whole-body weight loss, preferably about 1.8% over a population to whom the formulation was administered (“mean weight loss”), and/or a net change from placebo of about 3%, preferably about 2.7%. In some embodiments, administration thereof to a mammal results weight loss in an obese mammal sufficient to return the human being the normal weight range of a lean normal human being. In some embodiments, the pharmaceutical dosage form is an aqueous formulation comprising one or more of polysorbate 20, Arginine, or Mannitol.

In one aspect, a method for treatment of alcohol use disorder (AUD) in a human being in need thereof comprises administering to the human being a once weekly therapeutic effective amount of a pharmaceutical dosage formulation comprising a peptide product according to SEQ ID NO: 1, or a peptide product that is at least 80% identical to a peptide of SEQ ID NO: 1. In certain aspects, the peptide product is at least 85%, at least 90%, at least 95%, at least 96%, at least 97%, at least 98%, or at least 99% identical to a peptide of SEQ ID NO: 1. In certain aspects, the peptide product is 100% identical to a peptide of SEQ ID NO: 1. In certain aspects, the pharmaceutical dosage formulation is administered subcutaneously or by parenteral injection. In certain aspects, the pharmaceutical dosage is an aqueous formulation comprising one or more of polysorbate 20, arginine, or mannitol. In certain aspects, the pharmaceutical dosage formulation comprises about 0.025-0.5% (w/w) polysorbate 20, about 0.2-0.5% (w/w) arginine, and about 3-6% (w/w) mannitol in water (pH 7.7±0.1). In certain aspects, the pharmaceutical dosage formulation comprises about 0.2% (w/w) polysorbate 20, about 0.348% (w/w) arginine, and about 4.260% (w/w) mannitol in water (pH 7.7±0.1). In certain aspects, the human being is obese or overweight. In certain aspects, the human being in need thereof has a body mass index (BMI kg/m²) of at least 25. In certain aspects, the human being in need thereof has a body mass index (BMI kg/m²) of at least 30. In certain aspects, the human being in need thereof has a body mass index (BMI kg/m²) of at least 35. In certain aspects, the human being in need thereof has a body mass index (BMI kg/m²) of at least 40. In certain aspects, the human being in need thereof has a co-morbidity selected from one or more of fatty liver disease, hypertension, dyslipidemia, type 2 diabetes, polycystic ovary syndrome (PCOS), cardiovascular disease, hyperglycemia, and hyperinsulinemia. In certain aspects, the human being suffers from alcohol-related liver disease (ARLD). In certain aspects, the treatment reduces excess body weight. In certain aspects, the treatment reduces alcohol consumption by the human being. In certain aspects, the treatment maintains reduced alcohol consumption by the human being. In certain aspects, the treatment reduces phosphatidylethanol (PEth) levels in the human being. In certain aspects, the treatment reduces liver enzymes alanine aminotransferase (ALT) and/or aspartate aminotransferase (AST) in the human being. In certain aspects, the treatment reduces liver fat. In certain aspects, the weekly therapeutic effective amount of the pharmaceutical dosage formulation comprises 1.2 mg, 1.8 mg, or 2.4 mg of the peptide product. In certain aspects, the pharmaceutical dosage formulation comprises (2.4 mg/mL; 0.24% (w/w)) of the peptide product; mannitol (42.6 mg/mL; 4.26% (w/w)); arginine (3.48 mg/mL; 0.348% (w/w)); and polysorbate 20 (2 mg/mL; 0.2% (w/w)) in sterile water. In certain aspects, the pharmaceutical dosage formulation comprises (1.8 mg/mL; 0.18% (w/w)) of the peptide product; mannitol (42.6 mg/mL; 4.26% (w/w)); arginine (3.48 mg/mL; 0.348% (w/w)); and polysorbate 20 (2 mg/mL; 0.2% (w/w)) in sterile water. In certain aspects, the pharmaceutical dosage formulation comprises (1.2 mg/mL; 0.12% (w/w)) of the peptide product; mannitol (42.6 mg/mL; 4.26% (w/w)); arginine (3.48 mg/mL; 0.348% (w/w)); and polysorbate 20 (2 mg/mL; 0.2% (w/w)) in sterile water. In certain aspects, treatment reduces a symptom of withdrawal or prevents relapse of the alcohol use disorder in the human, reduces tolerance to alcohol, reduces dependence on alcohol, improves adherence to a treatment for alcohol abuse, reduces a preference for alcohol, decreases liking for alcohol, or increases abstinence from alcohol. In certain aspects, the method further comprises administering at least one additional therapeutic intervention selected from the group consisting of anti-craving medications such as naltrexone, acamprosate, or disulfiram, antidepressants, anxiolytics, and/or behavioral therapy.

In another aspect, a method for reducing alcohol consumption and/or maintaining reduced alcohol consumption in treatment of alcohol use disorder (AUD) in a human being in need thereof comprises administering to the human being a once weekly therapeutic effective amount of a pharmaceutical dosage formulation comprising a peptide product according to SEQ ID NO: 1, or a peptide product that is at least 80% identical to a peptide of SEQ ID NO: 1. In certain aspects, the peptide product is at least 85%, at least 90%, at least 95%, at least 96%, at least 97%, at least 98%, or at least 99% identical to a peptide of SEQ ID NO: 1. In certain aspects, the peptide product is 100% identical to a peptide of SEQ ID NO: 1. In certain aspects, the treatment reduces excess body weight. In certain aspects, the treatment reduces liver fat. In certain aspects, the human being in need thereof has a co-morbidity selected from one or more of fatty liver disease, hypertension, dyslipidemia, type 2 diabetes, polycystic ovary syndrome (PCOS), cardiovascular disease, hyperglycemia, and hyperinsulinemia. In certain aspects, the human being suffers from alcohol-related liver disease (ARLD). In certain aspects, the pharmaceutical dosage formulation is administered subcutaneously or by parenteral injection. In certain aspects, the pharmaceutical dosage is an aqueous formulation comprising one or more of polysorbate 20, arginine, or mannitol. In certain aspects, the pharmaceutical dosage formulation comprises about 0.025-0.5% (w/w) polysorbate 20, about 0.2-0.5% (w/w) arginine, and about 3-6% (w/w) mannitol in deionized water (pH 7.7±0.1). In certain aspects, the pharmaceutical dosage formulation comprises about 0.2% (w/w) polysorbate 20, about 0.348% (w/w) arginine, and about 4.260% (w/w) mannitol in deionized water (pH 7.7±0.1). In certain aspects, the human being is obese or overweight. In certain aspects, the human being in need thereof has a body mass index (BMI kg/m²) of at least 25. In certain aspects, the human being in need thereof has a body mass index (BMI kg/m²) of at least 30. In certain aspects, the human being in need thereof has a body mass index (BMI kg/m²) of at least 35. In certain aspects, the human being in need thereof has a body mass index (BMI kg/m²) of at least 40. In certain aspects, the treatment reduces phosphatidylethanol (PEth) levels in the human being. In certain aspects, the treatment reduces liver enzymes alanine aminotransferase (ALT) and/or aspartate aminotransferase (AST) in the human being. In certain aspects, the weekly therapeutic effective amount of the pharmaceutical dosage formulation comprises 1.2 mg, 1.8 mg, or 2.4 mg of SEQ ID NO: 1. In certain aspects, the pharmaceutical dosage formulation comprises (2.4 mg/mL; 0.24% (w/w)) of the peptide product; mannitol (42.6 mg/mL; 4.26% (w/w)); arginine (3.48 mg/mL; 0.348% (w/w)); and polysorbate 20 (2 mg/mL; 0.2% (w/w)) in sterile water. In certain aspects, the pharmaceutical dosage formulation comprises (1.8 mg/ml; 0.18% (w/w)) of the peptide product; mannitol (42.6 mg/mL; 4.26% (w/w)); arginine (3.48 mg/mL; 0.348% (w/w)); and polysorbate 20 (2 mg/mL; 0.2% (w/w)) in sterile water. In certain aspects, the pharmaceutical dosage formulation comprises (1.2 mg/mL; 0.12% (w/w)) of the peptide product; mannitol (42.6 mg/mL; 4.26% (w/w)); arginine (3.48 mg/mL; 0.348% (w/w)); and polysorbate 20 (2 mg/mL; 0.2% (w/w)) in sterile water. In certain aspects, treatment reduces a symptom of withdrawal or prevents relapse of the alcohol use disorder in the human, reduces tolerance to alcohol, reduces dependence on alcohol, improves adherence to a treatment for alcohol abuse, reduces a preference for alcohol, decreases liking for alcohol, or increases abstinence from alcohol.

Other aspects and embodiments are also contemplated herein as will be understood by those of ordinary skill in the art.

Unless defined otherwise or clearly indicated otherwise by their use herein, all technical and scientific terms used herein have the same meaning as commonly understood by those of ordinary skill in the art to which this application belongs. As used in the specification and the appended claims, the word “a” or “an” means one or more. As used herein, the word “another” means a second or more. The acronym “aka” means also known as. The term “exemplary” as used herein means “serving as an example, instance or illustration”. Any embodiment or feature characterized herein as “exemplary” is not necessarily to be construed as preferred or advantageous over other embodiments or features. In some embodiments, the term “about” or “approximately” means within ±10% or 5% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values. Ranges may be expressed herein as from about one particular value, and/or to about another particular value. When such a range is expressed, another aspect includes from the one particular value and/or to the other particular value. Similarly, when values are expressed as approximations, by use of the antecedent about or approximately, it will be understood that the particular value forms another aspect. It will be further understood that the endpoints of each of the ranges are significant both in relation to the other endpoint, and independently of the other endpoint. Ranges (e.g., 90-100%) are meant to include the range per se as well as each independent value within the range as if each value was individually listed. Optional or optionally means that the subsequently described event or circumstance can or cannot occur, and that the description includes instances where the event or circumstance occurs and instances where it does not. All publications, patents, and patent applications mentioned in this specification are herein incorporated by reference in their entireties to the same extent as if each individual publication, patent, or patent application was specifically and individually indicated to be incorporated by reference.

Certain embodiments are further described in the following examples. These embodiments are provided as examples only and are not intended to limit the scope of the claims in any way.

### EXAMPLES

### Example 1. Peptide Synthesis

The peptide and peptide products disclosed herein can be made in accordance with well-known procedures and by processes known and disclosed in the art. For example, SEQ ID NO: 1 can be prepared in accordance with synthetic protocols provided in U.S. Pat. No. 12,171,806. The contents of Example 1 of U.S. Pat. No. 12,171,806 are herein incorporated by reference.

### Example 2. Effects of SEQ ID NO: 1 on Chronic Ethanol Intake in Lean Golden

### Syrian Hamsters

The aim of this study was to evaluate the effects of SEQ ID NO: 1 (i.e., pemvidutide) on chronic ethanol intake in lean golden Syrian hamsters having a free choice between water and 15% (v/v) ethanol in water.

### Study Design:

The study included a 4-week induction period followed by a 3-week treatment period. For the duration of the study, hamsters (n=24) were provided ad libitum access to a standard chow diet and a free choice between normal tap water and 15% (v/v) ethanol in tap water. The induction period (Day-25 to Day-1) was intended to allow the hamsters to develop a preference for ethanol-containing water. During this phase, body weight, food intake, and fluid intake (tap water and 15% ethanol) were monitored three times per week. At the end of the induction phase, the animals were randomized into two groups (n=12 per group) based on body weight. The groups were treated for 3 weeks with either pemvidutide or vehicle control for 21 days. Pemvidutide (SEQ ID NO: 1) was administered daily via the subcutaneous route at a dose of 10 nmol/kg in a modified F58 buffer, which consisted of Polysorbate 20 (0.2% w/w), Arginine (0.348% w/w), Mannitol (4.26% w/w), and Water for Injection (pH 7.75). The vehicle control (modified F58 buffer only) was administered daily via the subcutaneous route. During the treatment period, body weight, food intake, and fluid intake (tap water and 15% ethanol) were monitored every two days. At the end of the study, the animals were euthanized, and livers were collected and weighed.

### Results:

During the induction period, water intake decreased progressively from 6.6 mL/hamster/day to 4 mL/hamster/day (39% decrease) while 15% ethanol intake increased from 1.5 mL/hamster/day to 7.8 mL/hamster/day (420% increase) indicating a marked preference toward ethanol (FIG. 1).

During the 3-week treatment period, pemvidutide induced a rapid and significant reduction in body weight consistent with the established mechanism of action of the drug (FIG. 2). At the end of the study, the vehicle group showed an average weight gain of 9.5 g (+7%) from Day 0, while the pemvidutide-treated group had an average weight loss of 19 g (−14%,) from Day 0.

Compared with vehicle, pemvidutide treatment led to a significant reduction in food intake (FIG. 3). While the vehicle treated group remained at an average food intake of 5.8 g/hamster/day throughout the study, the pemvidutide treated group showed a substantial reduction during the first 5 days of the treatment to 1.8 g/hamster/day by Day +5. After Day +5 of treatment, the food intake in the pemvidutide-treated group rebounded to an average food intake of 4.1 g/hamster/day at the end of the 3-week treatment period, but it remained significantly lower compared to the vehicle group.

At the end of the study, liver samples were collected, and the pemvidutide-treated group showed a significant reduction in total liver weight with an average decrease of 1.9 g, representing a 33.2% reduction in liver weight (FIG. 4A). The liver weight relative to body weight was also significantly reduced by pemvidutide treatment (FIG. 4B).

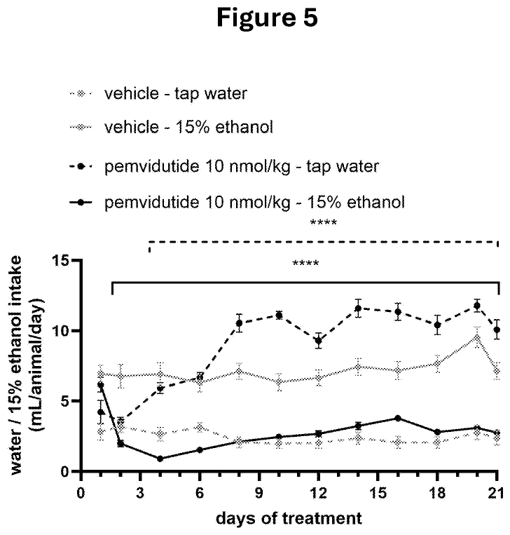

When observing fluid intake during the 3-week treatment period (FIG. 5), vehicle-treated hamsters showed a relatively stable intake of normal tap water and 15% ethanol (range of 2.4-2.8 mL/hamster/day for tap water, and 6.9-7.2 mL/hamster/day for 15% ethanol). In contrast, pemvidutide-treated hamsters showed a progressive and significant increase in normal tap water intake from ~4 mL/hamster/day to ~10 mL/hamster/day (+150%, p<0.001) between Day +1 and Day +21. Parallelly, pemvidutide treatment markedly repressed intake of 15% ethanol from 6.2 mL/hamster/day at Day +1 to 2.8 mL/hamster/day at Day +21 (−55%, p<0.001). The reduction was maximal by treatment Day +3 and was sustained during the 3-week treatment period.

Percentage ethanol preference over total fluid intake was also calculated for the two groups (FIG. 6). Results show that pemvidutide reduced ethanol preference within just 2 days of treatment, and this effect persisted through the end of the treatment period. No significant change in ethanol preference was observed in the vehicle group.

### Conclusion:

In the present hamster model of chronic ethanol intake, pemvidutide (SEQ ID NO: 1) showed a significant reduction in ethanol intake and significantly increased normal tap water intake compared to vehicle. In addition, Pemvidutide treated hamsters also had significant reductions in food intake, body weight and liver weight compared to vehicle. These effects suggest that pemvidutide may decrease ethanol-craving and ethanol use in humans suffering from Alcohol Use Disorder (AUD).

### Example 3. Phase 2, Multicenter, Randomized, Double-blind, Placebo-controlled Study Evaluating the Efficacy and Safety of Pemvidutide in the Treatment of Alcohol Use Disorder (AUD) in Subjects with Obesity or Overweight

This is a Phase 2, multicenter, randomized, double-blind, placebo-controlled study to evaluate the efficacy and safety of pemvidutide in the treatment of Alcohol Use Disorder (AUD) in obesity or overweight subjects a diagnosis of moderate or greater severity of AUD according to DSM-5. Inclusion criteria include: 1) written informed consent signed prior to performance of any study procedures; 2) male or female ages 18 to 75 years, inclusive; 3) diagnosis of current AUD of moderate or greater severity according to DSM-5 criteria; 4) reported drinking at least 28 drinks per week if male or 21 drinks per week if female in the 28 days prior to signing the informed consent (this should include at least 3 heavy drinking days per week (defined as ≥5 drinks per day for men and ≥4 drinks per day for women (Note: Baseline heavy drinking days are determined by the TFLB method (28-day recall) drinking pattern collected at the initial screening visit); 5) overweight or obesity, defined as BMI≥27 kg/m²; 6) agreement not to initiate additional behavioral therapy or counseling other than Take Control during the study (note: prior behavioral therapy or counseling that is ongoing at study entry is permitted provided that it began 90 or more days before screening); and, 7) female subjects of childbearing potential with negative serum pregnancy (beta human chorionic gonadotropin) test who are not breastfeeding, do not plan to become pregnant during the study, and agree to use highly effective birth control if they have sexual intercourse with a male partner (i.e., oral contraceptives; contraceptive patches, implants, injections, and rings; intrauterine devices [IUD], both IUDs hormonally-impregnated and untreated) throughout the study and for at least 1 month after study completion OR female subjects of non-childbearing potential (ie, surgically [bilateral oophorectomy, hysterectomy, or tubal ligation] or naturally sterile (>12 consecutive months without menses); or post-menopausal (>12 consecutive months without menses confirmed by follicular stimulating hormone (FSH) in the post-menopausal period) or male subjects who have sexual intercourse with a female partner of childbearing potential anytime from the first dose of pemvidutide or placebo until 1 month after study completion must either be surgically sterile or have female partners who meet the requirements for female subjects. All male subjects must agree not to donate sperm from the first dose of pemvidutide or placebo until 3 months after the last dose of pemvidutide or placebo. Subjects who have a same-sex partner or a surgically sterile partner, or practice sexual abstinence (only if this is in line with the subject's current lifestyle) are also permitted. These criteria will assure sufficient baseline severity to detect change on the primary endpoint and the secondary endpoint based on WHO risk drinking levels. GLP-1 RAs have been associated with an incidence (1%-2%) of hypoglycemic events in clinical trials, most notably in individuals with type 2 diabetes (T2D) using adjunctive antidiabetic medications, and similar findings have been made with pemvidutide. To reduce the possibility of these events occurring during this study, subjects with T2D have been excluded from study participation. To mitigate the risk of drug-induced liver injury, liver function tests are monitored on a frequent basis and investigators are directed to follow the decision criteria for the timing and method of intervention in subjects who develop worsening abnormal liver function tests, if observed. The algorithm for following liver function abnormality is based on the FDA Guidance for Industry, Drug-Induced Liver Injury: Premarketing Clinical Evaluation (Error! Reference source not found.2009) and Error! Reference source not found. (2019). These guidelines provide best practices for close monitoring, assessment, and management of liver function abnormalities, cholestatic liver injuries, and evaluation for other non-drug related causes (e.g., acute viral, alcoholic, or autoimmune hepatitis). Criteria for holding and reinstating pemvidutide or placebo are also specified, which are based on individual subject's baseline liver tests. An algorithm has also been implemented to modify treatment for individuals who experience hyperglycemia as the result of the effects of GCG agonism.

After signing the informed consent form, subjects are screened and if eligible randomized 1:1 to 1 of the following 2 treatment arms: 1) pemvidutide 1.2 mg SC once weekly for 4 weeks, 1.8 mg SC once weekly for 4 weeks, and 2.4 mg SC once weekly for 16 weeks, in sequence; and, 2) placebo SC once weekly for 24 weeks. The randomization of subjects is stratified based on sex and the presence or absence of severe AUD according to the Diagnostic and Statistical Manual of Mental Disorders (DSM-5), which are anticipated to be important covariates in the determination of the primary endpoint. The study design and sample size are considered sufficient to detect a meaningful difference in the primary endpoint at Weeks 21-24. Furthermore, meaningful secondary endpoints, including changes in other assessments of alcohol consumption by timeline follow back (TLFB) method, serum biomarkers, and drinking behavior outcomes, and safety and tolerability are assessed in this population. Subjects' drinking goals are assessed at screening (reduce drinking vs. total abstinence from drinking). Subjects will participate in regularly scheduled visits and assessments during the study. Subjects are provided pemvidutide or placebo for administration at home with a diary for documentation of date/time of dosing or missed doses. Alcohol consumption is reported by the TLFB method every 4 weeks. Subjects will also undergo standardized alcohol counseling using Take Control, a computer-based counseling program. Pemvidutide or placebo are administered as described herein. Selected visits may occur via telehealth at the discretion of the Investigator if no procedures requiring visits to the clinic are scheduled. The Clinical Institute Withdrawal Assessment for Alcohol-Revised (CIWA-Ar) questionnaire is administered at each clinic visit. On the day of randomization (Day 1), the CIWA-Ar is performed by qualified medical personnel on all subjects prior to the subject being randomized. If a subject presents with a CIWA-Ar score ≥10 at any time during the study, the subject is discontinued from the pemvidutide or placebo arms and referred for medical management. Investigators follow the decision criteria for the timing and method of intervention in subjects who develop worsening abnormal liver function tests during the 24-week treatment period. Dosing modifications, including dose reduction, may be permitted using the criteria outlined herein. Preferably, the Medical Monitor is consulted before dose modification or treatment discontinuation for any reason. Subjects who permanently discontinue treatment are encouraged to remain in the study and continue the study assessments described herein. Subjects are assessed for the presence of T2D at screening. Subjects with a diagnosis of T2D at screening are excluded from the study. Subjects are educated on symptoms and treatment of hyperglycemia and hypoglycemia. Subjects are administered the Columbia-Suicide Severity Rating Scale (C-SSRS) at each visit after the commencement of treatment with the exception of the visit on Day 3 or 4. Subjects with responses indicative of active suicidal thoughts (e.g., answering “yes” to any of Questions 2 through 5 on the C-SSRS) at any visit should be withdrawn from the study and referred to a mental health professional. The C-SSRS may be administered at any point during the trial if suicidality is suspected. Drug screens are performed at screening and each visit after commencing treatment. Subjects are excluded from study participation during the screening and/or treatment period for a positive urine drug screen that cannot be explained by diet, cigarette smoking, or permitted and appropriately prescribed and used medications. Sparse blood samples are collected for pemvidutide PK to be combined with data from other studies for population PK and PK-PD modeling. Blood samples will also be collected to assess immunogenicity and to correlate efficacy with ADA titers. Subjects who fail screening may be rescreened one time at the discretion of the Medical Monitor. The safety of study subjects is overseen by an independent Data Monitoring Committee (iDMC). In addition to routine safety surveillance, the iDMC is responsible for adjudicating AESIs reported during the study. Details of the role and responsibilities of the iDMC are described in the iDMC Charter.

Previous Phase I and Phase II clinical programs have explored single subcutaneous (SC) doses of pemvidutide up to 4.8 mg and once weekly SC dosing up to 2.4 mg. The available data demonstrate that once-weekly SC weekly doses of 1.2, 1.8, and 2.4 (titrated over four weeks) mg pemvidutide for 48 weeks were pharmacologically active, producing dose-dependent reductions in body weight, serum lipids, and liver fat content, and with maximal effects observed at the 2.4 mg dose. All three doses were also well-tolerated, with no significant differences in safety observations when the three doses were compared. Based on these findings, the 2.4 mg dose was selected for this trial to assess the maximum possible treatment effect while maintaining safety in this proof-of-concept study. Population PK modeling performed using combined data from Studies ALT-801-101, ALT-801-104, ALT-801-105/ALT-801-106, and ALT-801-211 also demonstrated strong correlations between body weight decrease and pemvidutide exposure, supporting the increased efficacy of higher doses. Pemvidutide exposure was also highly correlated with changes in liver fat and serum lipids. No significant association was observed between serum concentration of pemvidutide and glycemic parameters (HbA1c, fasting glucose, insulin, or HOMA-IR).

This study includes an extended 8-week titration schema to allow for improved tolerability of treatment while still providing a convenient dosing regimen for patients and prescribers. This 8-week titration schema is notably shorter than the titration periods employed with other incretin-containing agents, which span up to 24 weeks. Based on serum half-life and drug accumulation (see Investigator Brochure), steady state is expected to be achieved within three doses of commencing the 2.4 mg dose. In addition, subjects in this study are allowed dose reduction for intolerability. This will likely reduce treatment discontinuations and improve retention.

Thus, this study is designed to assess the assess the effects of pemvidutide on the severity of self-reported heavy alcohol consumption in healthy overweight and obese subjects (BMI 25.0-40.0 kg/m²) and to characterize the effective dose range based on pharmacokinetic-pharmacodynamic (PK-PD) relationships. Overweight and obese healthy volunteers are studied as the PK in such subjects may be different from that in normal weight individuals. In addition, these subjects are able to better tolerate the predicted PD effect of weight loss and could even benefit from treatment. Appropriate contraceptive measures have been put in place to minimize the chances of pregnancy, and precautions have been taken to exclude pre-existing conditions that would make subjects at risk for treatment with GLP-1 or glucagon analogues. Diabetic subjects have been excluded until the effects of pemvidutide on glucose homeostasis are better characterized in a non-diabetic population. As overweight and obese subjects typically exhibit varying levels of insulin resistance, the observations made in these studies, taken together with data from other compounds in this class, should be predictive of the effects observed when diabetic subjects are studied. Exclusions have been instituted that might otherwise affect an accurate assessment of the effects of ALT-801 on safety, PK, or PD. Analyses are conducted to evaluate the effects of the range of BMIs employed in this study on PK and PD parameters. The study shows the effects of ALT-801 on body weight, providing support for its use as a primary treatment for obesity.

The primary objective of the study is to assess the assess the effects of pemvidutide on the severity of self-reported heavy alcohol consumption in patients. The secondary objectives of the study are to evaluate the effects of pemvidutide on: 1) World Health Organization (WHO) risk level of self-reported alcohol consumption; 2) serum biomarkers of alcohol consumption; 3) self-reported alcohol consumption as assessed by frequency of daily drinking, sustained abstinence from heavy drinking, and complete abstinence from heavy drinking; 4) personal assessment of drinking behavior outcome as assessed by the Drinker Inventory of Consequences-Short Inventory of Problems (DrInc-SIP-2R), the Reported Outcomes Measurement Information System (PROMIS) Negative Alcohol Consequences Short Form (NECO), and anthropometric parameters including body weight, body mass index (BMI), and waist circumference. Exploratory objectives of the study include evaluation of the effects of pemvidutide on liver enzymes and tobacco smoking among smokers as assessed by self-reported tobacco use and serum cotinine levels. Safety is also evaluated by assessing the effects of pemvidutide on adverse events (AEs), vital signs, electrocardiograms (ECGs), and laboratory parameters in subjects with AUD. Population pharmacokinetics (PK) and pharmacokinetic-pharmacodynamic (PK-PD) relationships will also be determined.

Selected visits may occur via telehealth at the discretion of the Investigator if no procedures requiring visits to the clinic are scheduled. The Clinical Institute Withdrawal Assessment for Alcohol-Revised (CIWA-Ar) questionnaire is administered at each clinic visit. On the day of randomization (Day 1), the CIWA-Ar must be performed by qualified medical personnel on all subjects prior to the subject being randomized. If a subject presents with a CIWA-Ar score ≥10 at any time during the study, the subject is discontinued from the pemvidutide or placebo arms and referred for medical management. Investigators will follow the decision criteria for the timing and method of intervention in subjects who develop worsening abnormal liver function tests during the 24-week treatment period. Dosing modifications, including dose reduction, may be permitted for subjects who experience GI symptoms (e.g., nausea or vomiting) should be counseled on dietary behaviors (eg, eating smaller meals, splitting three daily meals into four or more smaller ones, and stopping eating when they feel full). If symptoms persist, the subject should be prescribed, at the Investigator's discretion, symptomatic medication (for example, antiemetic or antidiarrheal medication). If these or any symptoms persist or can no longer be tolerated, the Investigator should contact the Medical Monitor to discuss a dose reduction or dose interruption. In consultation with the Medical Monitor, a 2-week period of lowering of pemvidutide from 2.4 mg to 1.8 mg, from 1.8 mg to 1.2 mg, or from 1.2 mg to placebo for subjects randomized to pemvidutide, or from placebo to placebo for subjects randomized to placebo, may be considered for intolerable GI symptoms. After this period is completed, the subject should be encouraged to resume the prior dose. Subsequent dose reductions may be permitted in consultation with the Medical Monitor. Preferably, the Medical Monitor is consulted before dose modification or treatment discontinuation for any reason. Subjects who permanently discontinue treatment are encouraged to remain in the study and continue the study assessments delineated herein. Subjects are assessed for the presence of type 2 diabetes mellitus (T2D) at screening. Subjects with a diagnosis of T2D at screening are excluded from the study. Subjects are educated on symptoms and treatment of hyperglycemia and hypoglycemia. Subjects are administered the Columbia-Suicide Severity Rating Scale (C-SSRS) at each visit after the commencement of treatment with the exception of the visit on Day 3 or 4. Subjects with responses indicative of active suicidal thoughts (ie, answering “yes” to any of Questions 2 through 5 on the C-SSRS) at any visit should be withdrawn from the study and referred to a mental health professional. The C-SSRS may be administered at any point during the trial if suicidality is suspected. Drug screens are performed at screening and each visit after commencing treatment. Subjects are excluded from study participation during the screening and/or treatment period for a positive urine drug screen that cannot be explained by diet, cigarette smoking, or permitted and appropriately prescribed and used medications. Sparse blood samples are collected for pemvidutide PK to be combined with data from other studies for population PK and PK-PD modeling. Blood samples will also be collected to assess for immunogenicity and to correlate efficacy with anti-pemvidutide drug antibody (ADA) titers. Subjects who fail screening for laboratory values outside the acceptable range, elevated blood pressure, or non-allowed prior or concomitant medications may be rescreened one time at the discretion of the Medical Monitor. The safety of study subjects are overseen by an independent Data Monitoring Committee (iDMC). In addition to routine safety surveillance, the iDMC is responsible for adjudicating adverse events of special interest (AESIs) reported during the study. Details of the role and responsibilities of the iDMC are described in the iDMC Charter.

The Primary Efficacy Endpoint is the change from baseline in the average number of heavy drinking days per week, with a heavy drinking day defined as 5 or more drinks in the day for men and 4 or more drinks in the day for women, using the Timeline Followback (TLFB) method for the 4-week period comprising Weeks 21 through 24. Secondary Efficacy Endpoints include the proportion of subjects achieving a 2-level reduction in WHO drinking level using TLFB method for the 4-week period comprising Weeks 21 through 24, absolute change from baseline in average phosphatidylethanol (PEth) levels at Week 24, changes from baseline for the 4-week period comprising Week 21 through 24 self-reported using the TLFB method for the following measures of alcohol consumption (average number of drinks per drinking day (DDD), proportion of subjects achieving no heavy drinking days, and average percent days completely abstinent (PDA)), change from baseline at Week 24 in the following personal assessments of the adverse consequences of drinking (Average DrInc-SIP-2R, PROMIS NECO), and percent changes from baseline in each of the following anthropometric parameters at Week 24 (body weight, BMI, waist circumference). Exploratory Endpoints include change in liver enzymes, as assessed by range from baseline in aspartate aminotransferase (AST) at Week 24 in subjects with AST≥20 IU/L at baseline and change from baseline in ALT at Week 24 in subjects with ALT≥30 IU/L at baseline; and, change in tobacco use among smokers, as assessed by range in average self-reported tobacco use from baseline at Week 24 and change in average cotinine levels from baseline at Week 24. Safety Endpoints include frequency and severity of treatment-emergent adverse events (TEAEs), including, but not limited to: alcohol withdrawal symptoms, GI tolerability (nausea, vomiting, diarrhea, constipation); hyperglycemia and hypoglycemia; adverse events of special interest (AESIs; including but not limited to pancreatitis and MACE-3 [nonfatal stroke, nonfatal myocardial infarction, or cardiovascular death]); injection site reactions; changes in glucose homeostasis (HbA1c, fasting glucose, fasting insulin, Homeostatic Marker for Insulin Resistance [HOMA-IR]); changes in vital signs, including heart rate, systolic and diastolic blood pressure, rate-pressure product (RPP, calculated as mean heart rate×mean systolic pressure); changes in laboratory test values, including liver function tests, fasting glucose, amylase, lipase; changes in 12-lead ECG and ECG intervals; and immunogenicity (ADA titers).

The study will show that the Primary Efficacy Endpoint, and at least one or more of the Secondary Efficacy Endpoints, are met. Thus, the study will show that pemvidutide can be used in the treatment of human beings for alcohol use disorder to prevent and/or treat alcohol use disorder (AUD) and including treatment of disease/disorders associated with AUD and those considered co-morbidities of AUD.

In embodiments provided herein is a method for treatment of alcohol use disorder (AUD) and/or alcohol dependence in a human being in need thereof comprising administering to the human being a once weekly therapeutic effective amount of a pharmaceutical dosage formulation comprising a peptide product according to SEQ ID NO: 1 (pemvidutide), or a peptide product that is at least 80% identical to a peptide of SEQ ID NO: 1. In certain other embodiments provided herein is a method for reducing alcohol consumption and/or maintaining reduced alcohol consumption in treatment of alcohol use disorder (AUD) and/or alcohol dependence in a human being in need thereof comprising administering to the human being a once weekly therapeutic effective amount of a pharmaceutical dosage formulation comprising a peptide product according to SEQ ID NO: 1, or peptide product that is at least 80% identical to a peptide of SEQ ID NO: 1. In embodiments provided herein are methods for prevention of relapse to alcohol dependence in a human being in need thereof comprising administering to the human being a once weekly therapeutic effective amount of a pharmaceutical dosage formulation comprising a peptide product according to SEQ ID NO: 1, or peptide product that is at least 80% identical to a peptide of SEQ ID NO: 1.

Other advantages of the reagents and methods of using the same are also provided herein, as would be understood by those of ordinary skill in the art. While certain embodiments have been described in terms of the preferred embodiments, it is understood that variations and modifications will occur to those skilled in the art. Therefore, it is intended that the appended claims cover all such equivalent variations that come within the scope of the following claims.

1. A method for treatment of alcohol use disorder (AUD) and/or alcohol dependence in a human being in need thereof comprising administering to the human being a once weekly therapeutic effective amount of a pharmaceutical dosage formulation comprising a peptide product according to SEQ ID NO: 1, or a peptide product that is at least 80% identical to a peptide of SEQ ID NO: 1.

2. The method of claim 1, wherein the peptide product is at least 85%, at least 90%, at least 95%, at least 96%, at least 97%, at least 98% or at least 99% identical to a peptide of SEQ ID NO: 1.

3. The method of claim 1, wherein the peptide product is 100% identical to a peptide of SEQ ID NO: 1.

4. (canceled)

5. (canceled)

6. (canceled)

7. (canceled)

8. The method of claim 1, wherein the human being is obese or overweight.

9. (canceled)

10. (canceled)

11. (canceled)

12. (canceled)

13. The method of claim 1, wherein the human being in need thereof has a co-morbidity selected from one or more of fatty liver disease, hypertension, dyslipidemia, type 2 diabetes, polycystic ovary syndrome (PCOS), cardiovascular disease, hyperglycemia, and hyperinsulinemia.

14. The method of claim 1, wherein the human being suffers from alcohol-related liver disease (ARLD).

15. The method of claim 1, wherein the treatment reduces excess body weight.

16. The method of claim 1, wherein the treatment reduces alcohol consumption by the human being.

17. The method of claim 1, wherein the treatment maintains reduced alcohol consumption by the human being.

18. The method of claim 1, wherein the treatment reduces phosphatidylethanol (PEth) levels in the human being.

19. The method of claim 1, wherein the treatment reduces liver enzymes alanine aminotransferase (ALT) and/or aspartate aminotransferase (AST) in the human being.

20. The method of claim 1, wherein the treatment reduces liver fat.

21. The method of claim 1, wherein the weekly therapeutic effective amount of the pharmaceutical dosage formulation comprises about 1.2 mg, 1.8 mg or 2.4 mg of the peptide product.

22. (canceled)

23. (canceled)

24. (canceled)

25. The method of claim 1, wherein treatment reduces a symptom of withdrawal or prevents a relapse of the alcohol use disorder in the human, reduces tolerance to alcohol, reduces dependence on alcohol, improves adherence to a treatment for alcohol abuse, reduces a preference for alcohol, decreases liking for alcohol, or increases abstinence from alcohol.

26. The method of claim 1, further comprising administering at least one additional therapeutic intervention selected from the group consisting of: (a) anti-craving medications (such as naltrexone, acamprosate, or disulfiram), (b) antidepressants, (c) anxiolytics, and/or (d) behavioral therapy.

27. A method for reducing alcohol consumption and/or maintaining reduced alcohol consumption in treatment of alcohol use disorder (AUD) and/or alcohol dependence in a human being in need thereof comprising administering to the human being a once weekly therapeutic effective amount of a pharmaceutical dosage formulation comprising a peptide product according to SEQ ID NO: 1, or peptide product that is at least 80% identical to a peptide of SEQ ID NO: 1.

28-48. (canceled)

49. The method of claim 27, wherein the treatment reduces a symptom of withdrawal or prevents a relapse of the alcohol use disorder in the human, reduces tolerance to alcohol, reduces dependence on alcohol, improves adherence to a treatment for alcohol abuse, reduces a preference for alcohol, decreases liking for alcohol, or increases abstinence from alcohol.

50. A method for prevention of relapse to alcohol dependence in a human being in need thereof comprising administering to the human being a once weekly therapeutic effective amount of a pharmaceutical dosage formulation comprising a peptide product according to SEQ ID NO: 1, or peptide product that is at least 80% identical to a peptide of SEQ ID NO: 1.

51-55. (canceled)

56. The method of claim 50, wherein the human being suffers from alcohol-related liver disease (ARLD).

57-71. (canceled)

72. The method of claim 50, wherein treatment reduces a symptom of withdrawal or prevents a relapse of the alcohol use disorder in the human, reduces tolerance to alcohol, reduces dependence on alcohol, improves adherence to a treatment for alcohol abuse, reduces a preference for alcohol, decreases liking for alcohol, or increases abstinence from alcohol.


SEQUENCE LISTING (1 sequence)

SEQ ID NO: 1 - protein, 29 aa, synthetic construct
HXQGTFTSDYSKYLDEKAAKEFIQWLLQT